SEQUENCE LISTING (119 sequences)

SEQ ID NO: 1 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 2 - protein, 417 aa, Homo sapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 3 - protein, 405 aa, Homo sapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 4 - protein, 113 aa, Homo sapiens
QSVLTQPPSASGTPGQRVTISCSGSSSNIGNNYVSWYQQLPGTAPKLLIYRNNQRPSGVPDRFSGSKSGTSASLAISGLRSEDEADYYCQSYDSDLSGPYVLFGGGTKLTVLG

SEQ ID NO: 5 - protein, 111 aa, Homo sapiens
QSVLTQPPSASGTPGQRVTISCTGSSSNIGAGYDVHWYQQLPGTAPKLLIYGNSNRPSGVPDRFSGSKSGTSASLAISGLRSEDEAAYYCSSYVGSDLVVFGGGTKLTVLG

SEQ ID NO: 6 - protein, 112 aa, Homo sapiens
QSVLTQPPSTSGTPGQRVTISCTGSSSNIGAGFDVHWYQQLPGTAPKLLIYGNSNRPSGVPDRFSGSKSGTSASLAISGLRSEDEADYYCATWQDTLTGWMFGGGTKLTVLG

SEQ ID NO: 7 - protein, 110 aa, Homo sapiens
QSVLTQPPSASGTPGQRVTISCSGSDSNIGSNAVNWYQQLPGTAPKLLIYDNNKRPSGVPDRFSGSKSGTSASLAISGLRSEDEADYYCSSYTSSNTVVFGGGTKLTVLG

SEQ ID NO: 8 - protein, 112 aa, Homo sapiens
QSVLTQPPSASGTPGQRVTISCTGSSSNIGAAYDVHWYQQLPGTAPKLLIYGNNKRPSGVPDRFSGSKSGTSASLAISGLRSEDEADYYCQSYDSSLSGSVFGGGTKLTVLG

SEQ ID NO: 9 - protein, 112 aa, Homo sapiens
QSVLTQPPSASGTPGQRVTISCSGSSSNIRSNTVNWYQQLPGTAPKLLIYGNSNRPSGVPDRFSGSKSGTSASLAISGLRSEDEADYYCQSYDSSLSGDVVFGGGTKLTVLG

SEQ ID NO: 10 - protein, 121 aa, Homo sapiens
LTQPPSASGTPGQRVTISCTGSSSNIGAGYVVHWYQQLPGTAPKLLIYRNNHRPSGVPDRFSGSKSGTSASLAISGLRSEDEADYYCAAWDDSLNGRVFGGGTKLTVLGQPKAAPSVTLFP

SEQ ID NO: 11 - protein, 118 aa, Homo sapiens
LTQPPSASGTPGQRVTISCSGSSSNIGNNAVNWYQQLPGTAPKLLIYSNNQRPSGVPDRFSGSKSGTSASLAISGLRSEDEADYYCSSYTSSSTHVVFGGGTKLTVLGQPKAAPSVTL

SEQ ID NO: 12 - protein, 121 aa, Homo sapiens
LTQPPSASGTPGQRVTISCTGTSSNIGAGYDVHWYQQLPGTAPKLLIYDNNNRPSGVPDRFSGSKSGTSASLAISGLRSEDEADYYCAAWDDSLNGVVFGGGTKLTVLGQPKAAPSVTLFP

SEQ ID NO: 13 - protein, 120 aa, Homo sapiens
LTQPPSASGTPGQRVTISCTGSSSNIGAGYDVHWYQQLPGTAPKLLIYGNINRPSGVPDRFSGSKSGTSASLAISGLRSEDEADYYCSSYTRSATLVFGGGTKLTVLGQPKAAPSVTLFP

SEQ ID NO: 14 - protein, 124 aa, Homo sapiens
EVQLLESGGGLVQPGGSLRLSCAASGFTFGNHWMTWVRQAPGKGLEWVSGVSWNGSRTHYADSVKGRFTISRDNSKNTLYLQMNSLRAEDTAVYYCALTGRSGWMRFPNWFDPWGQGTLVTVTS

SEQ ID NO: 15 - protein, 124 aa, Homo sapiens; identical to SEQ ID NO: 14

SEQ ID NO: 16 - protein, 122 aa, Homo sapiens
EVQLLESGGGLVQPGGSLRLSCAASGFTFSSYSMNWVRQAPGKGLEWVSAISGSGGSTYYADSVKGRFTISRDNSKNTLYLQMNSLRAEDTAVYYCARDRRVRGIYDAFDMWGQGTLVTVTS

SEQ ID NO: 17 - protein, 124 aa, Homo sapiens
EVQLLESGGGLVQPGGSLRLSCAASGFTFSSNYMSWVRQAPGKGLEWVAVISYDGSNKYYADSVKGRFTISRDNSKNTLYLQMNSLRAEDTAMYYCALTGRSGWMRFPNWFDPWGQGTLVTVTS

SEQ ID NO: 18 - protein, 124 aa, Homo sapiens
EVQLLESGGGLVQPGGSLRLSCAASGFTFSSYWMSWVRQAPGKGLEWVSGVSWNGSRTHYADSVKGRFTISRDNSKNTLYLQMNSLRAEDTAVYYCALTGRSGWMRFPNWFDPWGQGTLVTVTS

SEQ ID NO: 19 - protein, 115 aa, Homo sapiens
EVQLLESGGGLVQPGGSLRLSCAASGFTFSSYGMHWVRQAPGKGLEWVSVIYSGGSTYYADSVKGRFTISRDNSKNTLYLQMNSLRAEDTAVYYCARMGRAFDIWGQGTLVTVTS

SEQ ID NO: 20 - protein, 126 aa, Homo sapiens
EVQLLESGGGLVQPGGSLRLSCAASGFTFSNYAMSWVRQSPGKGLEWVAVISYDGREKYYSDSVKGRFTISRDNSKNTLYLQMNSLRAEDTAVYYCARDRGRTFDYWGQGTLVTVTSASTKGPSVF

SEQ ID NO: 21 - protein, 133 aa, Homo sapiens
EVQLLESGGGLVQPGGSLRLSCAASGFTFNNYAMTWVRQAPGKGLEWVSGVSWNGSRTHYADSVKGRFTISRDNSKNTLYLQMNSLRAEDTAVYYCARADSSSAGRWAGSLDYWGQGTLVTVTSASTKGPSVF

SEQ ID NO: 22 - protein, 133 aa, Homo sapiens
EVQLLESGGGLVQPGGSLRLSCAASGFTFGNHWMTWVRQAPGKGLEWVSGVSWNGSRTHYADSVKGRFTISRDNSKNTLYLQMNSLRAEDTAVYYCALTGRSGWMRFPNWFDPWGQGTLVTVTSASTKGPSVF

SEQ ID NO: 23 - protein, 131 aa, Homo sapiens
EVQLLESGGGLVQPGGSLRLSCAASGFTFSGYGMHWVRQAPGKGLEWVSGINWNGGSTGYADSVKGRFTISRDNSKNTLYLQMNSLRAEDTAVYYCARNRATRSGYYYFDSWGQGTLVTVTSASTKGPSVF

SEQ ID NO: 24 - DNA, 339 bp, Homo sapiens
cagtctgtgctgactcagccaccctcagcgtctgggacccccgggcagagggtcaccatctcctgctctggaagcagctccaacattgggaataattatgtatcctggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgccagtcctatgacagcgacctgagtggtccctatgtgcttttcggcggaggaaccaagctgacggtcctaggt

SEQ ID NO: 25 - DNA, 333 bp, Homo sapiens
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgcttattactgcagctcatatgtaggcagcgacctcgtggtgttcggcggaggaaccaagctgacggtcctaggt

SEQ ID NO: 26 - DNA, 336 bp, Homo sapiens
cagtctgtgctgactcagccaccctcaacgtctgggacccccgggcagagggtcaccatctcctgcactgggagcagctccaacatcggggcaggttttgatgtacactggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgtgctacttggcaagacactctgactggttggatgttcggcggaggaaccaagctgacggtcctaggt

SEQ ID NO: 27 - DNA, 330 bp, Homo sapiens
cagtctgtgctgactcagccaccctcagcgtctgggacccccgggcagagggtcaccatctcttgttctggaagcgactccaacatcggaagtaatgctgttaattggtatcagcagctcccaggaacggcccccaaactcctcatctatgacaataataagcgaccctcaggggtccctgaccgattctctggctccaagtctggcacctcagcctccctggccatcagtgggctccggtccgaggatgaggctgattattactgcagctcatatacaagcagcaacactgtcgtattcggcggaggaaccaagctgacggtcctaggt

SEQ ID NO: 28 - DNA, 336 bp, Homo sapiens
cagtctgtgctgactcagccaccctcagcgtctgggacccccgggcagagggtcaccatctcctgcactgggagcagctccaacatcggggcagcttatgatgtacactggtatcagcagctcccaggaacggcccccaaactcctcatctatggcaataataagcgaccctcaggggtccctgaccgattctctggctccaagtctggcacctcagcctccctggccatcagtgggctccggtccgaggatgaggctgattattactgccagtcctatgacagcagcctgagtggttcggtattcggcggaggaaccaagctgacggtcctaggt

SEQ ID NO: 29 - DNA, 336 bp, Homo sapiens; identical to SEQ ID NO: 28

SEQ ID NO: 30 - DNA, 336 bp, Homo sapiens
cagtctgtgctgactcagccaccctcagcgtctgggacccccgggcagagggtcaccatctcctgcactgggagcagctccaacattggggcgggttatgttgtacattggtatcagcagctcccaggaacggcccccaaactcctcatctataggaataatcatcggccctcaggggtccctgaccgattctctggctccaagtctggcacctcagcctccctggccatcagtgggctccggtccgaggatgaggctgattattactgtgcagcatgggatgacagcctgaatggtcgggtgttcggcggaggaaccaagctgacggtcctaggt

SEQ ID NO: 31 - DNA, 333 bp, Homo sapiens
cagtctgtgctgactcagccaccctcagcgtctgggacccccgggcagagggtcaccatctcctgttctggaagcagctccaacatcggaaataatgctgtaaactggtatcagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgcagctcatatacaagcagcagcactcatgtggtattcggcggaggaaccaagctgacggtcctaggt

SEQ ID NO: 32 - DNA, 336 bp, Homo sapiens
cagtctgtgctgactcagccaccctcagcgtctgggacccccgggcagagggtcaccatctcctgcactgggaccagctccaacatcggggcaggttatgatgtacactggtatcagcagctcccaggaacggcccccaaactcctcatctatgataacaacaatcggccctcaggggtccctgaccgattctctggctccaagtctggcacctcagcctccctggccatcagtgggctccggtccgaggatgaggctgattattactgtgcagcatgggatgacagcctgaacggtgtggtattcggcggaggaaccaagctgacggtcctaggt

SEQ ID NO: 33 - DNA, 333 bp, Homo sapiens
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatggtaacatcaatcggccctcaggggtccctgaccgattctctggctccaagtctggcacctcagcctccctggccatcagtgggctccggtccgaggatgaggctgattattactgcagctcatatacaagaagcgccactctcgtgttcggcggaggaaccaagctgacggtcctaggt

SEQ ID NO: 34 - DNA, 372 bp, Homo sapiens
gaggtgcagctgttggagtctgggggaggcttggtacagcctggggggtccctgagactctcctgtgcagcctctggattcacctttggtaatcattggatgacctgggtccgccaggctccagggaaggggctggagtgggtatcgggtgttagttggaatggcagtaggacgcactatgcagactctgtgaagggccgattcaccatctccagagacaattccaagaacacgctgtatctgcaaatgaacagcctgagagccgaggacactgccgtgtattactgtgccctgacagggagaagtggctggatgcgcttcccaaactggttcgacccctggggccaaggtaccctggtcaccgtgactagt

SEQ ID NO: 35 - DNA, 372 bp, Homo sapines; identical to SEQ ID NO: 34

SEQ ID NO: 36 - DNA, 366 bp, Homo sapiens
gaggtgcagctgttggagtctgggggaggcttggtacagcctggggggtccctgagactctcctgtgcagcctctggattcacctttagtagttatagcatgaactgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacactgccgtgtattactgtgcgagagatcgacgggttcggggaatctatgatgcatttgatatgtggggccagggtaccctggtcaccgtgactagt

SEQ ID NO: 37 - DNA, 372 bp, Homo sapiens
gaggtgcagctgttggagtctgggggaggcttggtacagcctggggggtccctgagactctcctgtgcagcctctggattcaccttcagtagcaactacatgagctgggtccgccaggctccagggaaggggctggagtgggtggcagttatatcatatgatggaagcaataaatactacgcagactccgtgaagggccgattcaccatctccagagacaattccaagaacacgctgtatctgcaaatgaacagcctgagagccgaggacactgccatgtattactgtgccctgacagggagaagtggctggatgcgcttcccaaactggttcgacccctggggccaaggtaccctggtcaccgtgactagt

SEQ ID NO: 38 - DNA, 372 bp, Homo sapiens
gaggtgcagctgttggagtctgggggaggcttggtacagcctggggggtccctgagactctcctgtgcagcctctggattcacctttagtagctattggatgagctgggtccgccaggctccagggaaggggctggagtgggtatcgggtgttagttggaatggcagtaggacgcactatgcagactctgtgaagggccggttcaccatctccagagacaattccaagaacacgctgtatctgcaaatgaacagcctgagagccgaggacactgccgtgtattactgtgccctgacagggagaagtggctggatgcgcttcccaaactggttcgacccctggggccagggtaccctggtcaccgtgactagt

SEQ ID NO: 39 - DNA, 345 bp, Homo sapiens
gaggtgcagctgttggagtctgggggaggcttggtacagcctggggggtccctgagactctcctgtgcagcctctggattcaccttcagtagctatggcatgcactgggtccgccaggctccaggcaaggggctggagtgggtctcagttatttatagcggtggtagcacatactacgcagactccgtgaagggcagattcaccatctccagagacaattccaagaacacgctgtatctgcaaatgaacagcctgagagccgaggacactgccgtgtattactgtgcgaggatgggaagggcttttgatatctggggccaaggtaccctggtcaccgtgactagt

SEQ ID NO: 40 - DNA, 351 bp, Homo sapiens
gaggtgcagctgttggagtctgggggaggcttggtacagcctggggggtccctgagactctcctgtgcagcctctggattcacctttagcaactatgcgatgagctgggtccgccagtctccagggaaggggctggagtgggtggctgttatatcatatgacggaagggagaagtactactcagactccgtcaagggacgattcaccatctccagagacaattccaagaacacgctgtatctgcaaatgaacagcctgagagccgaggacactgccgtgtattactgtgcgagagatcggggacgtacctttgactactggggccaaggtaccctggtcaccgtgactagt

SEQ ID NO: 41 - DNA, 372 bp, Homo sapiens
gaggtgcagctgttggagtctgggggaggcttggtacagcctggggggtccctgagactctcctgtgcagcctctggattcacctttaacaactatgccatgacctgggtccgccaggctccagggaaggggctggagtgggtatcgggtgttagttggaatggcagtaggacgcactatgcagactctgtgaagggccgattcaccatctccagagacaattccaagaacacgctgtatctgcaaatgaacagcctgagagccgaggacactgccgtgtattactgtgcgagagcggatagcagcagcgccgggaggtgggccggctcacttgactactggggccaaggtaccctggtcaccgtgactagt

SEQ ID NO: 42 - DNA, 372 bp, Homo sapiens; identical to SEQ ID NO: 34

SEQ ID NO: 43 - DNA, 366 bp, Homo sapiens
gaggtgcagctgttggagtctgggggaggcttggtacagcctggggggtccctgagactctcctgtgcagcctctggattcaccttcagtggctatggcatgcactgggtccgccaggctccaggcaaggggctggagtgggtctctggtattaattggaatggtggtagcacaggttatgcagactctgtgaagggccgattcaccatctccagagacaattccaagaacacgctgtatctgcaaatgaacagcctgagagccgaggacactgccgtgtattactgtgcgagaaatcgagccacccgcagtggctactattactttgactcctggggccagggtaccctggtcaccgtgactagt

SEQ ID NO: 44 - protein, 13 aa, Homo sapiens
SGSSSNIGNNYVS

SEQ ID NO: 45 - protein, 14 aa, Homo sapiens
TGSSSNIGAGYDVH

SEQ ID NO: 46 - protein, 14 aa, Homo sapiens
TGSSSNIGAGFDVH

SEQ ID NO: 47 - protein, 13 aa, Homo sapiens
SGSDSNIGSNAVN

SEQ ID NO: 48 - protein, 14 aa, Homo sapiens
TGSSSNIGAAYDVH

SEQ ID NO: 49 - protein, 13 aa, Homo sapiens
SGSSSNIRSNTVN

SEQ ID NO: 50 - protein, 14 aa, Homo sapiens
TGSSSNIGAGYVVH

SEQ ID NO: 51 - protein, 13 aa, Homo sapiens
SGSSSNIGNNAVN

SEQ ID NO: 52 - protein, 14 aa, Homo sapiens
TGTSSNIGAGYDVH

SEQ ID NO: 53 - protein, 14 aa, Homo sapiens
TGSSSNIGAGYDVH

SEQ ID NO: 54 - protein, 6 aa, Homo sapiens
RNNQRP

SEQ ID NO: 55 - protein, 6 aa, Homo sapiens
GNSNRP

SEQ ID NO: 56 - protein, 6 aa, Homo sapiens
GNSNRP

SEQ ID NO: 57 - protein, 6 aa, Homo sapiens
DNNKRP

SEQ ID NO: 58 - protein, 6 aa, Homo sapiens
GNNKRP

SEQ ID NO: 59 - protein, 6 aa, Homo sapiens
GNSNRP

SEQ ID NO: 60 - protein, 6 aa, Homo sapiens
RNNHRP

SEQ ID NO: 61 - protein, 6 aa, Homo sapiens
SNNQRP

SEQ ID NO: 62 - protein, 6 aa, Homo sapiens
DNNNRP

SEQ ID NO: 63 - protein, 6 aa, Homo sapiens
GNINRP

SEQ ID NO: 64 - protein, 13 aa, Homo sapiens
QSYDSDLSGPYVL

SEQ ID NO: 65 - protein, 10 aa, Homo sapiens
SSYVGSDLVV

SEQ ID NO: 66 - protein, 11 aa, Homo sapiens
ATWQDTLTGWM

SEQ ID NO: 67 - protein, 10 aa, Homo sapiens
SSYTSSNTVV

SEQ ID NO: 68 - protein, 11 aa, Homo sapiens
QSYDSSLSGSV

SEQ ID NO: 69 - protein, 12 aa, Homo sapiens
QSYDSSLSGDVV

SEQ ID NO: 70 - protein, 11 aa, Homo sapiens
AAWDDSLNGRV

SEQ ID NO: 71 - protein, 11 aa, Homo sapiens
SSYTSSSTHVV

SEQ ID NO: 72 - protein, 11 aa, Homo sapiens
AAWDDSLNGVV

SEQ ID NO: 73 - protein, 10 aa, Homo sapiens
SSYTRSATLV

SEQ ID NO: 74 - protein, 5 aa, Homo sapiens
NHWMT

SEQ ID NO: 75 - protein, 5 aa, Homo sapiens
NHWMT

SEQ ID NO: 76 - protein, 5 aa, Homo sapiens
SYSMN

SEQ ID NO: 77 - protein, 5 aa, Homo sapiens
SNYMS

SEQ ID NO: 78 - protein, 5 aa, Homo sapiens
SYWMS

SEQ ID NO: 79 - protein, 5 aa, Homo sapiens
SYGMH

SEQ ID NO: 80 - protein, 5 aa, Homo sapiens
NYAMS

SEQ ID NO: 81 - protein, 5 aa, Homo sapiens
NYAMT

SEQ ID NO: 82 - protein, 5 aa, Homo sapiens
NHWMT

SEQ ID NO: 83 - protein, 5 aa, Homo sapiens
GYGMH

SEQ ID NO: 84 - protein, 18 aa, Homo sapiens
SGVSWNGSRTHYADSVKG

SEQ ID NO: 85 - protein, 18 aa, Homo sapiens
SGVSWNGSRTHYADSVKG

SEQ ID NO: 86 - protein, 18 aa, Homo sapiens
SAISGSGGSTYYADSVKG

SEQ ID NO: 87 - protein, 18 aa, Homo sapiens
AVISYDGSNKYYADSVKG

SEQ ID NO: 88 - protein, 18 aa, Homo sapiens
SGVSWNGSRTHYADSVKG

SEQ ID NO: 89 - protein, 17 aa, Homo sapiens
SVIYSGGSTYYADSVKG

SEQ ID NO: 90 - protein, 18 aa, Homo sapiens
AVISYDGREKYYSDSVKG

SEQ ID NO: 91 - protein, 18 aa, Homo sapiens
SGVSWNGSRTHYADSVKG

SEQ ID NO: 92 - protein, 18 aa, Homo sapiens
SGVSWNGSRTHYADSVKG

SEQ ID NO: 93 - protein, 18 aa, Homo sapiens
SGINWNGGSTGYADSVKG

SEQ ID NO: 94 - protein, 15 aa, Homo sapiens
TGRSGWMRFPNWFDP

SEQ ID NO: 95 - protein, 15 aa, Homo sapiens
TGRSGWMRFPNWFDP

SEQ ID NO: 96 - protein, 13 aa, Homo sapiens
DRRVRGIYDAFDM

SEQ ID NO: 97 - protein, 15 aa, Homo sapiens
TGRSGWMRFPNWFDP

SEQ ID NO: 98 - protein, 15 aa, Homo sapiens
TGRSGWMRFPNWFDP

SEQ ID NO: 99 - protein, 7 aa, Homo sapiens
MGRAFDI

SEQ ID NO: 100 - protein, 8 aa, Homo sapiens
DRGRTFDY

SEQ ID NO: 101 - protein, 15 aa, Homo sapiens
ADSSSAGRWAGSLDY

SEQ ID NO: 102 - protein, 15 aa, Homo sapiens
TGRSGWMRFPNWFDP

SEQ ID NO: 103 - protein, 13 aa, Homo sapiens
NRATRSGYYYFDS

SEQ ID NO: 104 - DNA, 25 bp, Artificial
  Primer Sequence
gaaacagctatgaaatacctattgc

SEQ ID NO: 105 - DNA, 19 bp, Artificial
  Primer Sequence
gcctgagcagtggaagtcc

SEQ ID NO: 106 - DNA, 24 bp, Artificial
  Primer Sequence
taggtatttcattatgactgtctc

SEQ ID NO: 107 - DNA, 23 bp, Artificial
  Primer Sequence
cccagtcacgacgttgtaaaacg

SEQ ID NO: 108 - protein, 217 aa, Homo sapiens
QSVLTQPPSASGTPGQRVTISCTGSSSNIGAAYDVHWYQQLPGTAPKLLIYGNNKRPSGVPDRFSGSKSGTSASLAISGLRSEDEADYYCQSYDSSLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 109 - protein, 453 aa, Homo sapiens
EVQLLESGGGLVQPGGSLRLSCAASGFTFSSYWMSWVRQAPGKGLEWVSGVSWNGSRTHYADSVKGRFTISRDNSKNTLYLQMNSLRAEDTAVYYCALTGRSGWMRFPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 217 aa, Homo sapiens
QSVLTQPPSVSGAPGQRVTISCTGSSSNIGAAYDVHWYQQLPGTAPKLLIYGNNKRPSGVPDRFSGSKSGTSASLAITGLQAEDEADYYCQSYDSSLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - protein, 449 aa, Homo sapiens
EVQLLESGGGLVQPGGSLRLSCAASGFTFSSYWMSWVRQAPGKGLEWVSGVSWGGSRTHYADSVKGRFTISRDNSKNTLYLQMNSLRAEDTAVYYCALTGRSGWMRFPNWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 112 - protein, 217 aa, Homo sapiens; identical to SEQ ID NO: 110

SEQ ID NO: 113 - protein, 449 aa, Homo sapiens
EVQLLESGGGLVQPGGSLRLSCAASGFTFSSYWMSWVRQAPGKGLEWVSGVSWQGSRTHYADSVKGRFTISRDNSKNTLYLQMNSLRAEDTAVYYCALTGRSGWMRFPNWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 114 - protein, 217 aa, Homo sapiens; identical to SEQ ID NO: 110

SEQ ID NO: 115 - protein, 449 aa, Homo sapiens
EVQLLESGGGLVQPGGSLRLSCAASGFTFSSYWMSWVRQAPGKGLEWVSGVSWAGSRTHYADSVKGRFTISRDNSKNTLYLQMNSLRAEDTAVYYCALTGRSGWMRFPNWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 116 - protein, 217 aa, Homo sapiens; identical to SEQ ID NO: 110

SEQ ID NO: 117 - protein, 449 aa, Homo sapiens
EVQLLESGGGLVQPGGSLRLSCAASGFTFSSYWMSWVRQAPGKGLEWVSGVSWNGARTHYADSVKGRFTISRDNSKNTLYLQMNSLRAEDTAVYYCALTGRSGWMRFPNWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 118 - protein, 217 aa, Homo sapiens; identical to SEQ ID NO: 110

SEQ ID NO: 119 - protein, 449 aa, Homo sapiens
EVQLLESGGGLVQPGGSLRLSCAASGFTFSSYWMSWVRQAPGKGLEWVSGVSWNGGRTHYADSVKGRFTISRDNSKNTLYLQMNSLRAEDTAVYYCALTGRSGWMRFPNWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

(19) United States
(12) Patent Application Publication
(10) Pub. No.: US 2015/0307625 A1
(43) Pub. Date: Oct. 29, 2015

(54) MONOCLONAL ANTIBODIES AGAINST ACTIVATED PROTEIN C (aPC)

(71) Applicant: BAYER HEALTHCARE LLC, Whippany, NJ (US)

(72) Inventors: Xiao-Yan ZHAO, Union City, CA (US); Zhuozhi WANG, Millbrae, CA (US); Jian-Ming GU, Lafayette, CA (US); Ji-Yun KIM, Berkeley, CA (US); Maxine BAUZON, Hercules, CA (US); John E. MURPHY, Berkeley, CA (US); Kirk MCLEAN, Orinda, CA (US); Fang JIN, Waban, MA (US); Tobias MARQUARDT, Wuppertal (DE); Xinquan WANG, Beijing (CN); Andreas WILMEN, Cologne (DE)

(73) Assignee: BAYER HEALTHCARE LLC, Whippany, NJ (US)

(21) Appl. No.: 14/443,710

(22) PCT Filed: Nov. 27, 2013
(86) PCT No.: PCT/US13/72243

§ 371 Date: May 19, 2015

Related U.S. Application Data
(60) Provisional application No. 61/731,294, filed on Nov. 29, 2012.
(60) Provisional application No. 61/786,472, filed on Mar. 15, 2013.

(51) Int. Cl. C07K 16/40; A61K 45/06; A61K 39/395

(52) U.S. Cl. CPC C07K 16/40; A61K 39/3955; A61K 45/06; C07K 2317/56; C07K 2317/76; C07K 2317/565; C07K 2317/30; A61K 2039/505

(57) ABSTRACT

Provided herein are antibodies, antigen-binding antibody fragments (Fabs), and other protein scaffolds, directed against human activated Protein C (aPC) with minimal binding to its zymogen Protein C (PC). Moreover, these aPC binding proteins could potentially block the anti-coagulant activity of aPC to induce coagulation. Therapeutic uses of these binders are described herein as are methods of panning and screening specific antibodies.

This application claims priority to U.S. Provisional Patent Application No. 61/731,294 filed Nov. 29, 2012 and to U.S. Provisional Patent Application No. 61/786,472 filed Mar. 15, 2013, the disclosures of which are hereby incorporated by reference herein in their entirety.

### SEQUENCE LISTING SUBMISSION

The Sequence Listing associated with this application is filed in electronic format via EFS-Web and hereby incorporated by reference into the specification in its entirety.

### FIELD OF THE EMBODIMENTS

Provided are isolated monoclonal antibodies and fragments thereof that preferentially bind activated form of human protein C (aPC).

### BACKGROUND

Human Protein C (PC) zymogen is synthesized in the liver as a 461-amino acid residue precursor and secreted into the blood (as shown in SEQ ID NO: 1). Prior to secretion, the single-chain polypeptide precursor is converted into a heterodimer by removal of a dipeptide (Lys156-Arg157) and a 42-aa residues preproleader. The heterodimeric form (417 residues) consists of the light chain (155aa, 21 kDa) and the heavy chain (262aa, 41 kDa) linked by a disulfide bridge (as shown in SEQ ID NO: 2). PC zymogen contains the thrombin cleavage site, leading to removal of the “activation peptide” and activation of PC to activated PC (aPC) form (405 residues) shown in SEQ ID NO: 3. FIG. 1 provides a cartoon depiction of human PC and its activated form, aPC. Human PC contains 9 Gla-residues and 4 potential sites for N-linked glycosylation. The light chain contains the Gla domain and 2 EGF-like domains. The heavy chain harbors an active serine protease domain.

PC normally circulates at 3-5 ug/ml (˜65 nM) in healthy human blood and its half-life is 6-8 hours. The predominant form of circulating PC zymogen is the heterodimeric form. The light chain of PC contains one gamma-carboxy glutamic acid (Gla)-rich domain (45aa), two EGF-like domains (46aa) and the linker sequences. The heavy chain of PC harbors a 12-aa highly polar “activation peptide” and a catalytic domain with a typical serine protease catalytic triad.

Human PC undergoes extensive post-translational modifications including glycosylation, vitamin K-dependent gamma-carboxylation, and gamma-hydroxylation (1-2). It contains 23% carbohydrate (by weight) and 4 potential N-linked glycosylation sites (one in the light chain Asn97 and three in the heavy chain Asn248/313/329). Its Gla domain contains 9 Gla residues and is responsible for the calcium-dependent binding of PC to negatively-charged phospholipid membranes. The Gla domain can also bind to the endothelial protein C receptor (EPCR), which aligns thrombin and thrombomodulin on the endothelial membrane during PC activation.

Protein C zymogen is typically converted to its active enzyme—activated protein C (aPC) to have biological potency. The activity of the PC pathway is controlled by the rate of PC activation and aPC inactivation. PC activation occurs on the surface of endothelial cells in a two-step process. It requires binding of PC (via Gla domain) to the EPCR on endothelial cells, followed by proteolytic activation of PC through thrombin/thrombomodulin complexes. A single cleavage at Arg12 of the heavy chain of human PC, which is catalyzed by thrombin/thrombomodulin on the endothelial cell surface, liberates the 12-aa AP and converts the zymogen PC into aPC, an active serine protease. Thus, the primary difference between the amino acid sequences of PC and aPC is the presence of a 12-aa activation peptide in PC that is absent in APC. Activation of PC into aPC also induces conformational changes; consequently only aPC, not PC, can be labeled by benzamidine or with chloromethylketone (CMK) peptide inhibitor in its enzymatic active site. The crystal structure of Gla-domainless aPC in complex with CMK-inhibitor was recently resolved. The major aPC inactivator in human plasma is the protein C inhibitor (PCI) present at 100 nM in human plasma, a member of the serpin superfamily. Under physiological conditions, aPC circulates at very low concentration (1-2 ng/ml or 40 pM) in human blood with a half-life of 20-30 min.

The protein C pathway serves as a natural defense mechanism against thrombosis. It differs from other anticoagulants in that it is an on-demand system that can amplify the anticoagulant response as the coagulant response increases. Upon injury, thrombin is generated for coagulation. At the same time, thrombin also triggers an anti-coagulant response by binding to thrombomodulin lined on the vascular surface, and this promotes protein C activation. Thus, aPC generation is roughly proportional to thrombin concentration and PC levels.

The physiological importance of the protein C pathway as a key regulator of coagulation process is shown by 3 clinical findings: (a) Severe thrombotic complications associated with protein C deficiency and the ability to correct the defect by protein C supplement (b) familial thrombophilia associated with deficiencies in protein C cofactor (protein S); and (c) thrombotic risk associated with the inherited mutations in its substrate (Factor V Leiden R506Q) which make it resistant to cleavage by aPC (Bernard, G R et. al. N Engl J Med 2001, 344:699-709 review).

In contrast to the other vitamin K-dependent coagulation factors, aPC functions as an anticoagulant by proteolytic inactivation of two coagulation cofactors, Factor Va and VIIIa, thereby inhibiting the generation of thrombin. As a result of decreased thrombin levels, the inflammatory, pro-coagulant and anti-fibrinolytic responses, induced by thrombin, are reduced. aPC also directly contributes to the enhanced fibrinolytic response by complex formation with plasminogen activator inhibitors (PAI).

In addition to its anti-coagulant functions, aPC induces cytoprotective effects, including anti-inflammatory and anti-apoptotic activities, and protection of endothelial barrier function. These direct cytoprotective effects of aPC on cells require EPCR and the G-protein-coupled receptor, protease activated receptor-1 (PAR-1). Thus, aPC promotes fibrinolysis and inhibits thrombosis and inflammation. The anti-coagulant and cytoprotective functions of aPC appear to be separable. Most of the cytoprotective effects are primarily independent of the anticoagulant activity of aPC and aPC mutants with minimal anti-coagulant activity and normal cytoprotective activity have been generated. Likewise, hyper-anticoagulant but non-cytoprotective aPC mutants have also been reported.

The C-terminus of aPC light chain is also a highly charged region that comprises residue Gly142-Leu155 on the opposite side of the active site in the protease domain. E149A-aPC had amidolytic activity that is indistinguishable from wild-typeaPC, but had more than a 3-fold increase in anti-coagulant activity in the activated partial thromboplastin time (aPTT) clotting assays due to increased sensitivity to protein S cofactor activity. E149A-aPC showed hyperactive anticoagulant activity in plasma-clotting assays as well as hyperactive anti-thrombotic potency in vivo. This mutant also had reduced cytoprotective and mortality reduction activities in a LPS-induced lethal endotoxemia murine model. This suggests that aPC's cytoprotective activity is required to reduce mortality in the murine model. In contrast, aPC's anticoagulant activity is neither necessary nor sufficient for mortality reduction. aPC has been used to treat sepsis, a life-threatening condition associated with hypercoagulation and generalized inflammatory reactions. A severe side effect of aPC therapy in sepsis is major bleeding that occurs in 2% of patients. This severe side effect limits its clinical use.

### SUMMARY

Monoclonal antibodies to human activated Protein C (aPC) are provided. In at least one embodiment, the anti-aPC monoclonal antibodies exhibit minimal binding to Protein C, which is the zymogen of aPC.

In some embodiments, the monoclonal antibodies to aPC provided have been optimized, for example to increase affinity, to increase functional activity or to reduce divergence from a germline sequence.

Also provided are specific epitopes on human aPC bound by isolated monoclonal antibody. Further provided are the isolated nucleic acid molecules encoding the same.

Pharmaceutical compositions comprising the anti-aPC monoclonal antibodies and methods of treatment of genetic and acquired deficiencies or defects in coagulation such as hemophilia A and B are also provided. Also provided are methods for shortening the bleeding time by administering an anti-aPC monoclonal antibody to a patient in need thereof. Methods for producing a monoclonal antibody that binds human aPC are also provided.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows a cartoon drawing of human activated Protein C in its mature heterodimer form.

FIG. 2 shows an amino acid sequence alignment of heavy and light chain CDRs is shown among 10 anti-aPC Fabs identified from the human Fab antibody library.

FIG. 3 depicts a graph characterizing anti-APC Fabs by direct ELISA. An ELISA plate was coated with human PC (hPC), human aPC (hAPC), dog aPC (dAPC), mouse aPC (mAPC) at 100 ng per well. Purified Fabs designate on the X-axis were added to the plate at 20 nM (1 ug/ml). Bound Fab was detected by the secondary antibody (anti-human Fab-HRP) followed by HRP substrate AmplexRed. The purified Fabs preferentially bind to human aPC and, with the exception of Fab R41C17, show little to no binding to human PC. One Fab T46J23 also showed some binding to mouse aPC.

FIG. 4 shows binding selectivity of anti-aPC Fabs by ELISA.

FIG. 5 depicts a graph showing inhibition of clot formation of normal human plasma in a dose-dependent manner by aPTT by spiking in human aPC. 50% pooled human normal plasma formed clots in 52 seconds. Preincubation of human aPC at 100, 200, 400, 800, or 1600 ng/ml with the plasma prolonged the clotting time in a dose-dependent manner. Nearly identical potency for recombinant human aPC (rh-APC) and plasma-derived human aPC (pdh-APC) was observed.

FIG. 6 depicts graphs showing anti-aPC Fabs inhibit human aPC and induce clot formation in human normal plasma. Human aPC at 400 ng/ml extended the plasma clotting time from 52 seconds to 180 seconds. Incubation of control antibody (Control) or its Fab (Control-Fab) or select Fabs at 0, 0.5, 1, 2, 5, 10, or 20 ug/ml with aPC reduced the clotting time in a dose-dependent manner (top panel). Three Fabs (R41E3, C22J13, Control-Fab) were also tested at 40 ug/ml for a greater effect (bottom panel).

FIG. 7 shows anti-aPC Fabs inhibit dog aPC and induce clot formation in aPTT.

FIG. 8 shows the effect of anti-aPC Fabs on the amidolytic activity of aPC. Human aPC protein (20 nM) was first preincubated with an equal volume of anti-aPC Fab (1-3000 nM) at room temperature for 20 min before the chromogenic substrate SPECTROZYME PCa was added to the reaction mixture up to 1 mM. The amidolytic activity of human aPC at a final concentration of 10 nM was measured in the presence of Fabs. Hydrolysis rates were inhibited in the presence of the Fabs, reaching a maximum reduction of 80%.

FIG. 9 shows the effect of anti-aPC Fabs on the Factor Va (FVa) inactivation activity of aPC.

FIG. 10 shows binding specificity of anti-aPC human IgG1s and shows species cross-reactivity of anti-aPC human IgG1s by ELISA. An ELISA plate was coated with human PC (hPC), human aPC (hAPC), dog aPC, mouse aPC, rabbit aPC at 1 ug/ml. Purified IgGs (20 nM) were added to the plate. Bound IgG was detected by the secondary antibody (anti-human IgG-HRP) followed by HRP substrate AmplexRed. Five anti-aPC human IgG1s cross-react with dog and rabbit aPCs and one IgG1 also binds mouse aPC.

FIG. 11 shows the effect of anti-aPC IgGs on amidolytic activity of species aPCs—(a) human, (b) rabbit, (c) dog, and (d) mouse. aPC protein (20 nM) was first preincubated with an equal volume of anti-aPC-hIgG1 (1-1000 nM) at room temperature for 20 min before the chromogenic substrate SPECTROZYME PCa was added to the reaction mixture up to 1 mM. The amidolytic activity of aPC at a final concentration of 10 nM was measured in the presence of Fabs. Hydrolysis rates were inhibited in the presence of the IgGs. A negative control antibody (anti-CTX-hIgG1) was used.

FIG. 12 shows anti-aPC-hIgG1s shorten clotting time and induce coagulation in human plasma clotting assays (aPTT).

FIG. 13 shows the effect of anti-aPC-IgG1 on severe hemophilic patient plasma. In the presence of endothelial cells and thrombomodulin, PC is activated to aPC and reduces thrombin generation. Unlike control Ab, anti-aPC-antibody rapidly inhibits this newly generated aPC and increase thrombin generation by 5-10×. Enhanced thrombin generation will lead to improved coagulation in patients with coagulopathy.

FIG. 14 shows activity profile of anti-aPC-antibody variants. Similar to the parental antibody, C25K23, such variants (a) bind to aPC with high affinity, (b) potently inhibit aPC activity in purified system, and (c) shorten clotting time leading to coagulation in human plasma clotting assay.

FIG. 15 shows a cartoon depicting the complex structure was refined to a final Rwork=0.201, Rfree=0.241. The left and right panels show the same complex structure with a rotation change of 90°. The HCDR3 loop from the Fab C25K23 has extensive interactions with the heavy chain of aPC.

FIG. 16 shows in the left panel shows a zoomed view of interactions around the residue Trp104 in the CDR3 loop of Fab C25K23 heavy chain. It blocks the accessibility of active site of aPC (catalytically important residues His57, Asp102, and Ser195). The right panel shows that the Fab C25K23 inhibits the activity of aPC in a way similar to the PPACK inhibitor because Trp104 and PPACK occupy the same region at the active site.

FIG. 17 shows a graph depicting anti-aPC antibodies, in both Fab and IgG forms, binding or not binding to active-site-blocked aPC by ELISA.

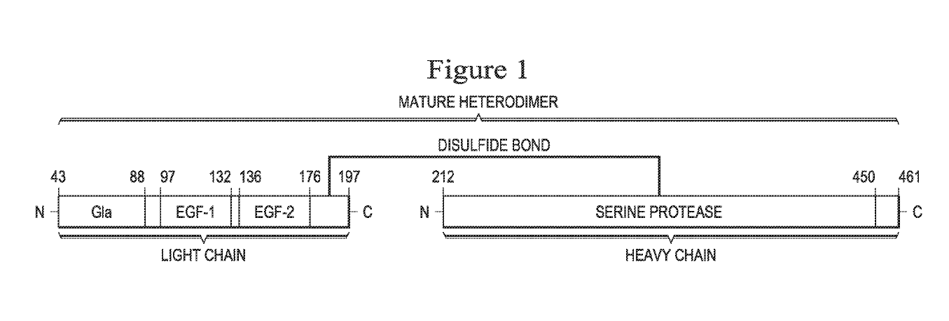

### DETAILED DESCRIPTION

As discussed above, the present disclosure provides antibodies, including monoclonal antibodies, and other binding proteins that specifically bind to the activated form of human Protein C (aPC), but exhibit comparatively little or no reactivity against the zymogen form of human Protein C (PC).

For purposes of this patent document, the following terminology will be used with the definitions set out below.

### DEFINITIONS

Whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and are not limiting. For example, the term “including” shall mean “including, but not limited to.”

The term “Protein C” or “PC” as used herein refers to any variant, isoform, and/or species homolog of Protein C in its zymogen form that is naturally expressed by cells and present in plasma and is distinct from the activated form of Protein C.

The term “activated Protein C” or “aPC” as used herein refers to an activated form of Protein C that is characterized by the absence of a 12 amino acid activation peptide present in Protein C.

As used herein, an “antibody” refers to a whole antibody and any antigen binding fragment (i.e., “antigen-binding portion”) or single chain thereof. The term includes a full-length immunoglobulin molecule (e.g., an IgG antibody) that is naturally occurring or formed by normal immunoglobulin gene fragment recombinatorial processes, or an immunologically active portion of an immunoglobulin molecule, such as an antibody fragment, that retains the specific binding activity. Regardless of structure, an antibody fragment binds with the same antigen that is recognized by the full-length antibody. For example, an anti-aPC monoclonal antibody fragment binds to an epitope of aPC. The antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR); (vii) minibodies, diaboidies, triabodies, tetrabodies, and kappa bodies (see, e.g. Ill et al., Protein Eng 1997; 10:949-57); (viii) camel IgG; and (ix) IgNAR. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are analyzed for utility in the same manner as are intact antibodies.

Furthermore, it is contemplated that an antigen binding fragment can be encompassed in an antibody mimetic. The term “antibody mimetic” or “mimetic” as used herein is meant a protein that exhibits binding similar to an antibody but is a smaller alternative antibody or a non-antibody protein. Such antibody mimetic can be comprised in a scaffold. The term “scaffold” refers to a polypeptide platform for the engineering of new products with tailored functions and characteristics.

As used herein, the term “anti-aPC antibody” refers to an antibody that specifically binds to an epitope of aPC. When bound in vivo to an epitope of aPC, the anti-aPC antibodies disclosed herein augment one or more aspects of the blood clotting cascade.

As used herein, the terms “inhibits binding” and “blocks binding” (e.g., referring to inhibition/blocking of binding of aPC substrate to aPC) are used interchangeably and encompass both partial and complete inhibition or blocking of a protein with its substrate, such as an inhibition or blocking by at least about 10%, about 20%, about 30%, about 40%, about 50%, about 60%, about 70%, about 80%, about 90%, about 95%, about 96%, about 97%, about 98%, about 99%, or about 100%. As used herein, “about” means +/−10% of the numerical value indicated.

In reference to the inhibition and/or blocking of binding of aPC substrate to aPC, the terms inhibition and blocking also include any measurable decrease in the binding affinity of aPC to a physiological substrate when in contact with an anti-aPC antibody as compared to aPC not in contact with an anti-aPC antibody, e.g., the blocking of the interaction of aPC with its substrates, including Factor Va or with Factor VIIIa, by at least about 10%, about 20%, about 30%, about 40%, about 50%, about 60%, about 70%, about 80%, about 90%, about 95%, about 96%, about 97%, about 98%, about 99%, or about 100%.

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity that have variable and constant regions derived from human germline immunoglobulin sequences. The human antibodies can include amino acid residues not encoded by human germline immunoglobulin sequences (e.g., mutations introduced by random or site-specific mutagenesis in vitro or by somatic mutation in vivo).

An “isolated antibody,” as used herein, is intended to refer to an antibody which is substantially free of other biological molecules, including antibodies having different antigenic specificities (e.g., an isolated antibody that binds to aPC is substantially free of antibodies that bind antigens other than aPC). In some embodiments, the isolated antibody is at least about 75%, about 80%, about 90%, about 95%, about 97%, about 99%, about 99.9% or about 100% pure by dry weight. In some embodiments, purity can be measured by a method such as column chromatography, polyacrylamide gel electrophoresis, or HPLC analysis. An isolated antibody that binds to an epitope, isoform or variant of human aPC can, however, have cross-reactivity to other related antigens, e.g., from other species (e.g., aPC species homologs). Moreover, an isolated antibody can be substantially free of other cellular material and/or chemicals. As used herein, “specific binding” refers to antibody binding to a predetermined antigen. Typically, an antibody that exhibits “specific binding” binds to an antigen with an affinity of at least about 105 M-1 and binds to that antigen with an affinity that is higher, for example at least two-fold greater, than its binding affinity for an irrelevant antigen (e.g., BSA, casein). The phrases “an antibody recognizing an antigen” and “an antibody specific for an antigen” are used interchangeably herein with the term “an antibody which binds specifically to an antigen.”

As used herein, the term “minimal binding” refers to an antibody that does not bind to and/or exhibits low affinity to a specified antigen. Typically, an antibody having minimal binding to an antigen binds to that antigen with an affinity that is lower than about 102 M-1 and does not bind to a predetermined antigen with higher affinity than it binds to an irrelevant antigen.

As used herein, the term “high affinity” for an antibody, such as an IgG antibody refers to a binding affinity of at least about 107M-1, in at least one embodiment at least about 108M-1, in some embodiments at least about 109M-1, 1010M-1, 1011M-1 or greater, e.g., up to 1013M-1 or greater. However, “high affinity” binding can vary for other antibody isotypes. For example, “high affinity” binding for an IgM isotype refers to a binding affinity of at least about 107M-1. As used herein, “isotype” refers to the antibody class (e.g., IgM or IgG1) that is encoded by heavy chain constant region genes.

“Complementarity-determining region” or “CDR” refers to one of three hypervariable regions within the variable region of the heavy chain or the variable region of the light chain of an antibody molecule that form the N-terminal antigen-binding surface that is complementary to the three-dimensional structure of the bound antigen. Proceeding from the N-terminus of a heavy or light chain, these complementarity-determining regions are denoted as “CDR1,” “CDR2,” and “CDR3,” respectively [Wu T T, Kabat E A, Bilofsky H, Proc Natl Acad Sci USA. 1975 December; 72(12):5107 and Wu T T, Kabat E A, J Exp Med. 1970 Aug. 1; 132(2):211]. CDRs are involved in antigen-antibody binding, and the CDR3 comprises a unique region specific for antigen-antibody binding. An antigen-binding site, therefore, can include six CDRs, comprising the CDR regions from each of a heavy and a light chain V region.

The term “epitope” refers to the area or region of an antigen to which an antibody specifically binds or interacts, which in some embodiments indicates where the antigen is in physical contact with the antibody. Conversely, the term “paratope” refers to the area or region of the antibody on which the antigen specifically binds. Epitopes characterized by competition binding are said to be overlapping if the binding of the corresponding antibodies are mutually exclusive, i.e. binding of one antibody excludes simultaneous binding of another antibody. The epitopes are said to be separate (unique) if the antigen is able to accommodate binding of both corresponding antibodies simultaneously.

The term “competing antibodies,” as used herein, refers to antibodies that bind to about, substantially or essentially the same, or even the same, epitope as an antibody against aPC as described herein. “Competing antibodies” include antibodies with overlapping epitope specificities. Competing antibodies are thus able to effectively compete with an antibody as described herein for binding to aPC. In some embodiments, the competing antibody can bind to the same epitope as the antibody described herein. Alternatively viewed, the competing antibody has the same epitope specificity as the antibody described herein.

As used herein, “conservative substitutions” refers to modifications of a polypeptide that involve the substitution of one or more amino acids for amino acids having similar biochemical properties that do not result in loss of a biological or biochemical function of the polypeptide. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Antibodies of the present disclosure can have one or more conservative amino acid substitutions yet retain antigen binding activity.

For nucleic acids and polypeptides, the term “substantial homology” indicates that two nucleic acids or two polypeptides, or designated sequences thereof, when optimally aligned and compared, are identical, with appropriate nucleotide or amino acid insertions or deletions, in at least about 80% of the nucleotides or amino acids, usually at least about 85%, in some embodiments about 90%, 91%, 92%, 93%, 94%, or 95%, in at least one embodiment at least about 96%, 97%, 98%, 99%, 99.1%, 99.2%, 99.3%, 99.4%, or 99.5% of the nucleotides or amino acids. Alternatively, substantial homology for nucleic acids exists when the segments will hybridize under selective hybridization conditions to the complement of the strand. Also included are nucleic acid sequences and polypeptide sequences having substantial homology to the specific nucleic acid sequences and amino acid sequences recited herein.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, such as without limitation the AlignX™ module of VectorNTI™ (Invitrogen Corp., Carlsbad, Calif.). For AlignX™, the default parameters of multiple alignment are: gap opening penalty: 10; gap extension penalty: 0.05; gap separation penalty range: 8; % identity for alignment delay: 40. (further details found at http://www.invitrogen.com/site/us/en/home/LINNEA-Online-Guides/LINNEA-CommunitiesNector-NTI-Community/Sequence-analysis-and-data-management-software-for-PCs/AlignX-Module-for-Vector-NTI-Advance.reg.us.html).

Another method for determining the best overall match between a query sequence (a sequence of the present disclosure) and a subject sequence, also referred to as a global sequence alignment, can be determined using the CLUSTALW computer program (Thompson et al., Nucleic Acids Research, 1994, 2(22): 4673-4680), which is based on the algorithm of Higgins et al., (Computer Applications in the Biosciences (CABIOS), 1992, 8(2): 189-191). In a sequence alignment the query and subject sequences are both DNA sequences. The result of said global sequence alignment is in percent identity. Parameters that can be used in a CLUSTALW alignment of DNA sequences to calculate percent identity via pairwise alignments are: Matrix=IUB, k-tuple=1, Number of Top Diagonals=5, Gap Penalty=3, Gap Open Penalty=10, Gap Extension Penalty=0.1. For multiple alignments, the following CLUSTALW parameters can be used: Gap Opening Penalty=10, Gap Extension Parameter=0.05; Gap Separation Penalty Range=8; % Identity for Alignment Delay=40.

The nucleic acids can be present in whole cells, in a cell lysate, or in a partially purified or substantially pure form. A nucleic acid is “isolated” or “rendered substantially pure” when purified away from other cellular components with which it is normally associated in the natural environment. To isolate a nucleic acid, standard techniques such as the following can be used: alkaline/SDS treatment, CsCl banding, column chromatography, agarose gel electrophoresis and others well known in the art.

### Monoclonal Antibodies Against Activated Protein C

aPC is known for its anti-coagulant properties. Bleeding disorders where homeostasis is deregulated in hemophilia or in trauma patients where the wound results in a temporary loss of hemostatsis, can be treated by aPC inhibitors. Antibodies, antigen-binding fragments thereof, and other aPC-specific protein scaffolds can be used to provide targeting specificity to inhibit a subset of aPC protein functions while preserving the rest. Given the at least 1000-fold difference in plasma concentration of aPC (<4 ng/ml) versus PC (4 ug/ml), increased specificity of any potential aPC inhibitor therapeutics is helpful to block aPC function in the presence of a high circulating excess of PC.

aPC specific antibodies that block the anti-coagulant function of aPC can be used as therapeutics for patients with bleeding disorders, including, for example, hemophilia, hemophilia patients with inhibitors, trauma-induced coagulopathy, severe bleeding patients during sepsis treatment by aPC, bleeding resulting from elective surgery such as transplantation, cardiac surgery, orthopedic surgery, or excessive bleeding from Menorrhagia.

Anti-aPC antibodies having long circulating half-live can be useful in treating chronic diseases like hemophilia. aPC antibody fragments or aPC-binding protein scaffolds with shorter half-lives can be more effective for acute use (e.g. therapeutic use in trauma). As aPC is a multi-function protein, selective aPC function blockers (SAFB) including antibodies, antigen-binding antibody fragments, aPC-specific protein scaffolds with increased affinity and targeting specificity can selectively block only one aPC function without affecting other aPC functions.

aPC-binding antibodies were identified by panning and screening human antibody libraries against human aPC. The identified antibodies exhibited no or minimal binding to human PC. The heavy chain variable region and light chain variable region of each monoclonal antibody isolated was sequenced and its CDR regions were identified. The sequence identifier numbers (“SEQ ID NO”) that correspond to the heavy and light chain regions of each of the aPC-specific monoclonal antibodies are summarized in Table 1.

TABLE 1
Human anti-aPC antibodies

| | Light Chain | SEQ | Heavy Chain | SEQ |
|---|---|---|---|---|
| Clone | Variable Region | ID | Variable Region | ID |
| C7A23 | QSVLTQPPSASGTPG | SEQ | EVQLLESGGGLVQPG | SEQ |
| | QRVTISCSGSSSNIG | ID | GSLRLSCAASGFTFG | ID |
| | NNYVSWYQQLPGTAP | NO: | NHWMTWVRQAPGKGL | NO: |
| | KLLIYRNNQRPSGVP | 4 | EWVSGVSWNGSRTHY | 14 |
| | DRFSGSKSGTSASLA | | ADSVKGRFTISRDNS | |
| | ISGLRSEDEADYYCQ | | KNTLYLQMNSLRAED | |
| | SYDSDLSGPYVLFGG | | TAVYYCALTGRSGWM | |
| | GTKLTVLG | | RFPNWFDPWGQGTLV | |
| | | | TVTS | |
| C7I7 | QSVLTQPPSASGTPG | SEQ | EVQLLESGGGLVQPG | SEQ |
| | QRVTISCTGSSSNIG | ID | GSLRLSCAASGFTFG | ID |
| | AGYDVHWYQQLPGTA | NO: | NHWMTWVRQAPGKGL | NO: |
| | PKLLIYGNSNRPSGV | 5 | EWVSGVSWNGSRTHY | 15 |
| | PDRFSGSKSGTSASL | | ADSVKGRFTISRDNS | |
| | AISGLRSEDEAAYYC | | KNTLYLQMNSLRAED | |
| | SSYVGSDLVVFGGGT | | TAVYYCALTGRSGWM | |
| | KLTVLG | | RFPNWFDPWGQGTLV | |
| | | | TVTS | |
| O3E7 | QSVLTQPPSTSGTPG | SEQ | EVQLLESGGGLVQPG | SEQ |
| | QRVTISCTGSSSNIG | ID | GSLRLSCAASGFTFS | ID |
| | AGFDVHWYQQLPGTA | NO: | SYSMNWVRQAPGKGL | NO: |
| | PKLLIYGNSNRPSGV | 6 | EWVSAISGSGGSTYY | 16 |
| | PDRFSGSKSGTSASL | | ADSVKGRFTISRDNS | |
| | AISGLRSEDEADYYC | | KNTLYLQMNSLRAED | |
| | ATWQDTLTGWMFGGG | | TAVYYCARDRRVRGI | |
| | TKLTVLG | | YDAFDMWGQGTLVTV | |
| | | | TS | |
| C22J13 | QSVLTQPPSASGTPG | SEQ | EVQLLESGGGLVQPG | SEQ |
| | QRVTISCSGSDSNIG | ID | GSLRLSCAASGFTFS | ID |
| | SNAVNWYQQLPGTAP | NO: | SNYMSWVRQAPGKGL | NO: |
| | KLLIYDNNKRPSGVP | 7 | EWVAVISYDGSNKYY | 17 |
| | DRFSGSKSGTSASLA | | ADSVKGRFTISRDNS | |
| | ISGLRSEDEADYYCS | | KNTLYLQMNSLRAED | |
| | SYTSSNTVVFGGGTK | | TAMYYCALTGRSGWM | |
| | LTVLG | | RFPNWFDPWGQGTLV | |
| | | | TVTS | |
| C25K23 | QSVLTQPPSASGTPG | SEQ | EVQLLESGGGLVQPG | SEQ |
| | QRVTISCTGSSSNIG | ID | GSLRLSCAASGFTFS | ID |
| | AAYDVHWYQQLPGTA | NO: | SYWMSWVRQAPGKGL | NO: |
| | PKLLIYGNNKRPSGV | 8 | EWVSGVSWNGSRTHY | 18 |
| | PDRFSGSKSGTSASL | | ADSVKGRFTISRDNS | |
| | AISGLRSEDEADYYC | | KNTLYLQMNSLRAED | |
| | QSYDSSLSGSVFGGG | | TAVYYCALTGRSGWM | |
| | TKLTVLG | | RFPNWFDPWGQGTLV | |
| | | | TVTS | |
| C26B9 | QSVLTQPPSASGTPG | SEQ | EVQLLESGGGLVQPG | SEQ |
| | QRVTISCSGSSSNIR | ID | GSLRLSCAASGFTFS | ID |
| | SNTVNWYQQLPGTAP | NO: | SYGMHWVRQAPGKGL | NO: |
| | KLLIYGNSNRPSGVP | 9 | EWVSVIYSGGSTYYA | 19 |
| | DRFSGSKSGTSASLA | | DSVKGRFTISRDNSK | |
| | ISGLRSEDEADYYCQ | | NTLYLQMNSLRAEDT | |
| | SYDSSLSGDVVFGGG | | AVYYCARMGRAFDIW | |
| | TKLTVLG | | GQGTLVTVTS | |
| R41C17 | LTQPPSASGTPGQRV | SEQ | EVQLLESGGGLVQPG | SEQ |
| | TISCTGSSSNIGAGY | ID | GSLRLSCAASGFTFS | ID |
| | VVHWYQQLPGTAPKL | NO: | NYAMSWVRQSPGKGL | NO: |
| | LIYRNNHRPSGVPDR | 10 | EWVAVISYDGREKYY | 20 |
| | FSGSKSGTSASLAIS | | SDSVKGRFTISRDNS | |
| | GLRSEDEADYYCAAW | | KNTLYLQMNSLRAED | |
| | DDSLNGRVFGGGTKL | | TAVYYCARDRGRTFD | |
| | TVLGQPKAAPSVTLF | | YWGQGTLVTVTSAST | |
| | P | | KGPSVF | |
| R41E3 | LTQPPSASGTPGQRV | SEQ | EVQLLESGGGLVQPG | SEQ |
| | TISCSGSSSNIGNNA | ID | GSLRLSCAASGFTFN | ID |
| | VNWYQQLPGTAPKLL | NO: | NYAMTWVRQAPGKGL | NO: |
| | IYSNNQRPSGVPDRF | 11 | EWVSGVSWNGSRTHY | 21 |
| | SGSKSGTSASLAISG | | ADSVKGRFTISRDNS | |
| | LRSEDEADYYCSSYT | | KNTLYLQMNSLRAED | |
| | SSSTHVVFGGGTKLT | | TAVYYCARADSSSAG | |
| | VLGQPKAAPSVTL | | RWAGSLDYWGQGTLV | |
| | | | TVTSASTKGPSVF | |
| T46J23 | LTQPPSASGTPGQRV | SEQ | EVQLLESGGGLVQPG | SEQ |
| | TISCTGTSSNIGAGY | ID | GSLRLSCAASGFTFG | ID |
| | DVHWYQQLPGTAPKL | NO: | NHWMTWVRQAPGKGL | NO: |
| | LIYDNNNRPSGVPDR | 12 | EWVSGVSWNGSRTHY | 22 |
| | FSGSKSGTSASLAIS | | ADSVKGRFTISRDNS | |
| | GLRSEDEADYYCAAW | | KNTLYLQMNSLRAED | |
| | DDSLNGVVFGGGTKL | | TAVYYCALTGRSGWM | |
| | TVLGQPKAAPSVTLF | | RFPNWFDPWGQGTLV | |
| | P | | TVTSASTKGPSVF | |
| T46P19 | LTQPPSASGTPGQRV | SEQ | EVQLLESGGGLVQPG | SEQ |
| | TISCTGSSSNIGAGY | ID | GSLRLSCAASGFTFS | ID |
| | DVHWYQQLPGTAPKL | NO: | GYGMHWVRQAPGKGL | NO: |
| | LIYGNINRPSGVPDR | 13 | EWVSGINWNGGSTGY | 23 |
| | FSGSKSGTSASLAIS | | ADSVKGRFTISRDNS | |
| | GLRSEDEADYYCSSY | | KNTLYLQMNSLRAED | |
| | TRSATLVFGGGTKLT | | TAVYYCARNRATRSG | |
| | VLGQPKAAPSVTLFP | | YYYFDSWGQGTLVTV | |
| | | | TSASTKGPSVF | |

In one embodiment, provided is an isolated monoclonal antibody that binds to human activated protein C (aPC) and inhibits anticoagulant activity but has minimal binding to unactivated Protein C, wherein the antibody comprises a heavy chain variable region comprising an amino acid sequence selected from the group consisting of SEQ ID Nos: 14-23.

In another embodiment, provided is an isolated monoclonal antibody that binds to human activated protein C (aPC) and inhibits anticoagulant activity but has minimal binding to unactivated Protein C, wherein the antibody comprises a light chain variable region comprising an amino acid sequence selected from the group consisting of SEQ ID Nos: 4-13.

In another embodiment, provided is an isolated monoclonal antibody that binds to human activated protein C (aPC) and inhibits anticoagulant activity but has minimal binding to unactivated Protein C, wherein the antibody comprises a heavy chain variable region comprising an amino acid sequence selected from the group consisting of SEQ ID Nos: 14-23 and a light chain variable region comprising an amino acid sequence selected from the group consisting of SEQ ID Nos: 4-13.

In other embodiments, the antibody comprises heavy and light chain variable regions comprising:

a heavy chain variable region comprising an amino acid sequence of SEQ ID NO: 14 and a light chain variable region comprising an amino acid sequence of SEQ ID NO: 4;

a heavy chain variable region comprising an amino acid sequence of SEQ ID NO: 15 and a light chain variable region comprising an amino acid sequence of SEQ ID NO: 5;

a heavy chain variable region comprising an amino acid sequence of SEQ ID NO: 16 and a light chain variable region comprising an amino acid sequence of SEQ ID NO: 6;

a heavy chain variable region comprising an amino acid sequence of SEQ ID NO: 17 and a light chain variable region comprising an amino acid sequence of SEQ ID NO: 7;

a heavy chain variable region comprising an amino acid sequence of SEQ ID NO: 18 and a light chain variable region comprising an amino acid sequence of SEQ ID NO: 8;

a heavy chain variable region comprising an amino acid sequence of SEQ ID NO: 19 and a light chain variable region comprising an amino acid sequence of SEQ ID NO: 9;

a heavy chain variable region comprising an amino acid sequence of SEQ ID NO: 20 and a light chain variable region comprising an amino acid sequence of SEQ ID NO: 10;

a heavy chain variable region comprising an amino acid sequence of SEQ ID NO: 21 and a light chain variable region comprising an amino acid sequence of SEQ ID NO: 11;

a heavy chain variable region comprising an amino acid sequence of SEQ ID NO: 22 and a light chain variable region comprising an amino acid sequence of SEQ ID NO: 12; and

a heavy chain variable region comprising an amino acid sequence of SEQ ID NO: 23 and a light chain variable region comprising an amino acid sequence of SEQ ID NO: 13.

Shown in Table 2 is a summary of the SEQ ID Nos for the CDR regions (“CDR1”, “CDR2”, and “CDR3”) of each heavy and light chain of the monoclonal antibodies binding to human aPC.

TABLE 2
Sequence Identifiers for CDR regions of Human Anti-aPC antibodies

| | Light Chain Variable Region | | | Heavy Chain Variable Region | | |
|---|---|---|---|---|---|---|
| Clone | CDR1 | CDR2 | CDR3 | CDR1 | CDR2 | CDR3 |
| C7A23 | 44 | 54 | 64 | 74 | 84 | 94 |
| C7I7 | 45 | 55 | 65 | 75 | 85 | 95 |
| O3E7 | 46 | 56 | 66 | 76 | 86 | 96 |
| C22J13 | 47 | 57 | 67 | 77 | 87 | 97 |
| C25K23 | 48 | 58 | 68 | 78 | 88 | 98 |
| C26B9 | 49 | 59 | 69 | 79 | 89 | 99 |
| R41C17 | 50 | 60 | 70 | 80 | 90 | 100 |
| R41E3 | 51 | 61 | 71 | 81 | 91 | 101 |
| T46J23 | 52 | 62 | 72 | 82 | 92 | 102 |
| T46P19 | 53 | 63 | 73 | 83 | 93 | 103 |

In one embodiment, provided is an isolated monoclonal antibody that binds to human activated protein C (aPC), wherein the antibody comprises a CDR3 comprising an amino acid sequence selected from the group consisting of SEQ ID NOs: 94-103. These CDR3s are identified from the heavy chains of the antibodies identified during panning and screening. In a further embodiment, this antibody further comprises (a) a CDR1 comprising an amino acid sequence selected from the group consisting of SEQ ID NOs: 74-83, (b) a CDR2 comprising an amino acid sequence selected from the group consisting of SEQ ID NOs: 84-93, or (c) both a CDR1 comprising an amino acid sequence selected from the group consisting of SEQ ID NOs: 74-83 and a CDR2 comprising an amino acid sequence selected from the group consisting of SEQ ID NOs: 84-93.

In another embodiment, provided are antibodies that share a CDR3 from one of the light chains of the antibodies identified during panning and screening. Thus, also provided is an isolated monoclonal antibody, wherein said antibody binds to activated Protein C and inhibits anticoagulant activity but has minimal binding to unactivated Protein C, wherein said antibody comprises a CDR3 comprising an amino acid sequence selected from the group consisting of SEQ ID NOs: 64-73. In further embodiments, the antibody further comprises (a) a CDR1 comprising an amino acid sequence selected from the group consisting of SEQ ID NOs: 44-53, (b) a CDR2 comprising an amino acid sequence selected from the group consisting of SEQ ID NOs: 54-63, or (c) both a CDR1 comprising an amino acid sequence selected from the group consisting of SEQ ID NOs: 44-53 and a CDR2 comprising an amino acid sequence selected from the group consisting of SEQ ID NOs: 54-63.

In another embodiment, the antibody comprises a CDR3 from a heavy chain and a light chain of the antibodies identified from screening and panning. Provided is an isolated monoclonal antibody, wherein said antibody binds to activated Protein C and inhibits anticoagulant activity but has minimal binding to unactivated Protein C, wherein said antibody comprises a CDR3 comprising an amino acid sequence selected from the group consisting of SEQ ID NOs: 94-103 and a CDR3 comprising an amino acid sequence selected from the group consisting of SEQ ID NOs: 64-73. In a further embodiment, the antibody further comprises (a) a CDR1 comprising an amino acid sequence selected from the group consisting of SEQ ID NOs: 74-83, (b) a CDR2 comprising an amino acid sequence selected from the group consisting of SEQ ID NOs: 84-93, (c) a CDR1 comprising an amino acid sequence selected from the group consisting of SEQ ID NOs: 44-53, and/or (d) a CDR2 comprising an amino acid sequence selected from the group consisting of SEQ ID NOs: 54-63.

In some embodiments, the antibody comprises heavy and light chain variable regions comprising:

a light chain variable region comprising an amino acid sequence comprising SEQ ID NOs: 44, 54, and 64 and a heavy chain variable region comprising an amino acid sequence comprising SEQ ID NOs: 74, 84, and 94;

a light chain variable region comprising an amino acid sequence comprising SEQ ID NOs: 45, 55, and 65 and a heavy chain variable region comprising an amino acid sequence comprising SEQ ID NOs: 75, 85, and 95;

a light chain variable region comprising an amino acid sequence comprising SEQ ID NOs: 46, 56, and 66 and a heavy chain variable region comprising an amino acid sequence comprising SEQ ID NOs: 76, 86, and 96;

a light chain variable region comprising an amino acid sequence comprising SEQ ID NOs: 47, 57, and 67 and a heavy chain variable region comprising an amino acid sequence comprising SEQ ID NOs: 77, 87, and 97;

a light chain variable region comprising an amino acid sequence comprising SEQ ID NOs: 48, 58, and 68 and a heavy chain variable region comprising an amino acid sequence comprising SEQ ID NOs: 78, 88, and 98;

a light chain variable region comprising an amino acid sequence comprising SEQ ID NOs: 49, 59, and 69 and a heavy chain variable region comprising an amino acid sequence comprising SEQ ID NOs: 79, 89, and 99;

a light chain variable region comprising an amino acid sequence comprising SEQ ID NOs: 50, 60, and 70 and a heavy chain variable region comprising an amino acid sequence comprising SEQ ID NOs: 80, 90, and 100;

a light chain variable region comprising an amino acid sequence comprising SEQ ID NOs: 51, 61, and 71 and a heavy chain variable region comprising an amino acid sequence comprising SEQ ID NOs: 81, 91, and 101;

a light chain variable region comprising an amino acid sequence comprising SEQ ID NOs: 52, 62, and 72 and a heavy chain variable region comprising an amino acid sequence comprising SEQ ID NOs: 82, 92, and 102; and

a light chain variable region comprising an amino acid sequence comprising SEQ ID NOs: 53, 63, and 73 and a heavy chain variable region comprising an amino acid sequence comprising SEQ ID NOs: 83, 93, and 103.

Also provided is an isolated monoclonal antibody that binds to activated Protein C and inhibits anticoagulant activity but has minimal binding to unactivated Protein C, wherein said antibody comprises an amino acid sequence having at least 89%, 90%, 91%, 92%, 93%, 94%, 95%, 96%, 97%, 98%, 99% or 99.5% identity to an amino acid sequence selected from the group consisting of the amino acid sequences set forth in SEQ ID NO: 4-13.

Also provided is an isolated monoclonal antibody that binds to activated Protein C and inhibits anticoagulant activity but has minimal binding to unactivated Protein C, wherein said antibody comprises an amino acid sequence having at least 89%, 90%, 91%, 92%, 93%, 94%, 95%, 96%, 97%, 98%, 99% or 99.5% identity to an amino acid sequence selected from the group consisting of the amino acid sequences set forth in SEQ ID NO: 14-23.

The antibody can be species specific or can cross react with multiple species. In some embodiments, the antibody can specifically react or cross react with aPC of human, mouse, rat, rabbit, guinea pig, monkey, pig, dog, cat or other mammalian species.

The antibody can be of any of the various classes of antibodies, such as without limitation an IgG1, an IgG2, an IgG3, an IgG4, an IgM, an IgA1, an IgA2, a secretory IgA, an IgD, and an IgE antibody.

In one embodiment, provided is an isolated fully human monoclonal antibody to human activated protein C.

Optimized Variants of Anti-aPC Antibodies

In some embodiments, the antibodies panned and screened can be optimized, for example to increase affinity to aPC, to further decrease any affinity to PC, to improve cross-reactivity to different species, or to improve blocking activity of aPC. Such optimization can be performed for example by utilizing site saturation mutagenesis of the CDRs or amino acid residues in close proximity to the CDRs, i.e. about 3 or 4 residues adjacent to the CDRs, of the antibodies.

Also provided are monoclonal antibodies having increased or high affinity to aPC. In some embodiments, the anti-aPC antibodies have a binding affinity of at least about 107M-1, in some embodiments at least about 108M-1, in some embodiments at least about 109M-1, 1010M-1, 1011M-1 or greater, e.g., up to 1013M-1 or greater.

In some embodiments, additional amino acid modifications can be introduced to reduce divergence from the germline sequence. In other embodiments, amino acid modifications can be introduced to facilitate antibody production for large scale production processes.

In some embodiments, provided are isolated anti-aPC monoclonal antibodies that specifically bind to human activated Protein C, which antibodies comprise one or more amino acid modifications. In some embodiments, the antibody comprises 1, 2, 3, 4, 5, 6, 7, 8, 9, 10, 11, 12, 13, 14, 15, 16, 17, 18, 19, or 20 or more modifications.

Accordingly, in some embodiments, provided is an isolated monoclonal antibody that binds to human activated Protein C, wherein the antibody comprises a light chain comprising the amino acid sequence shown in SEQ ID NO: 8, wherein the amino acid sequence comprises one or more amino acid modifications. In some embodiments, the modification of the light chain is a substitution, an insertion or a deletion. In some embodiments, the modifications are located in the CDRs of the light chain. In other embodiments, the modifications are located outside the CDRs of the light chain.

In some embodiments, the modification of the light chain of SEQ ID NO:8 is at a position selected from G52, N53, N54, R56, P57, S58, Q91, Y93, S95, S96, L97, S98, G99, S100 and V101. The modification can be for example one of the following substitutions: G52S, G52Y, G52H, G52F, N53G, N54K, N54R, R56K, P57G, P57W, P57N, S58V, S58F, S58R, Q91R, Q91G, Y93W, S95F, S95Y, S95G, S95W, S95E, S96G, S96A, S96Y, S96W, S96R, L97M, L97G, L97R, L97V, S98L, S98W, S98V, S98R, G99A, G99E, S100A, S100V, V101Y, V101L or V101E. Further, in some embodiments, the antibody may comprise two or more substitutions from G52S, G52Y, G52H, G52F, N53G, N54K, N54R, R56K, P57G, P57W, P57N, S58V, S58F, S58R, Q91R, Q91G, Y93W, S95F, S95Y, S95G, S95W, S95E, S96G, S96A, S96Y, S96W, S96R, L97M, L97G, L97R, L97V, S98L, S98W, S98V, S98R, G99A, G99E, S100A, S100V, V101Y, V101L or V101E.

In some embodiments, the light chain of SEQ ID NO:8 further comprises a modification at one or more of the positions selected from A10, T13, S78, R81 and S82. In some embodiments, the modification at position A10 in the light chain is A10V. In some embodiments, the modification at position T13 in the light chain is T13A. In some embodiments, the modification at position S78 in the light chain is S78T. In some embodiments, the modification at position R81 in the light chain is R81Q. In some embodiments, the modification at position S82 in the light chain is S82A. In some embodiments, the light chain of SEQ ID NO:8 comprises two or more of the modifications A10V, T13A, S78T, R81Q and S82A. In some embodiments, the light chain of SEQ ID NO:8 comprises all the modifications A10V, T13A, S78T, R81Q and S82A.

In other embodiments, provided is an isolated monoclonal antibody that specifically binds to human activated form of Protein C, wherein the antibody comprises a heavy chain having the amino acid sequence shown in SEQ ID NO: 18, wherein the amino acid sequence comprises one or more amino acid modifications. In some embodiments, the modification of the light chain is a substitution, an insertion or a deletion.

In some embodiments, the heavy chain of SEQ ID NO:18 further comprises a modification at positions N54 or S56. In some embodiments, the modification at position N54 of the heavy chain is N54G, N54Q or N54A. In some embodiments, modification at position S56 of the heavy chain is S56A or S56G.

In some embodiments, amino acid modifications can be made in order to in order to facilitate antibody production for large scale production processes. For example, in some embodiments, modifications can be made to reduce the hydrophobic surface region of antibodies for improved biophysical properties (e.g. minimal aggregation/stickiness). In some embodiments, additional modifications are made in the light chain of SEQ ID NO: 8. In some embodiments, the modification of the light chain of SEQ ID NO:8 is at position Y33. In some embodiments, the modification and Y33 in the light chain is Y33A, Y33K or Y33D. In some embodiments, additional modifications are made in the heavy chain of SEQ ID NO:18. In some embodiments, the modifications of the heavy chain of SEQ ID NO:18 are at one or more of the positions Y32, W33, W53 or W110. In some embodiments, the modification in the heavy chain of SEQ ID NO:18 is selected from Y32A, Y32K, Y32D, W33A, W33K, W33D, W53A, W53K, W53D, W110A, W110K, or W110D.

In some embodiments, provided is an isolated monoclonal antibody that binds to human activated Protein C, wherein the antibody comprises a light chain having the amino acid sequence shown in SEQ ID NO: 108. In some embodiments, provided is an isolated monoclonal antibody that binds to human activated Protein C, wherein the antibody comprises a light chain having the amino acid sequence shown in SEQ ID NO: 110. In some embodiments, provided is an isolated monoclonal antibody that binds to human activated Protein C, wherein the antibody comprises a light chain having the amino acid sequence shown in SEQ ID NO: 112. In some embodiments, provided is an isolated monoclonal antibody that binds to human activated Protein C, wherein the antibody comprises a light chain having the amino acid sequence shown in SEQ ID NO: 114. In some embodiments, provided is an isolated monoclonal antibody that binds to human activated Protein C, wherein the antibody comprises a light chain having the amino acid sequence shown in SEQ ID NO: 116. In some embodiments, provided is an isolated monoclonal antibody that binds to human activated Protein C, wherein the antibody comprises a light chain having the amino acid sequence shown in SEQ ID NO: 118.

In some embodiments, provided is an isolated monoclonal antibody that binds to human activated Protein C, wherein the antibody comprises a heavy chain having the amino acid sequence shown in SEQ ID NO: 109. In some embodiments, provided is an isolated monoclonal antibody that binds to human activated Protein C, wherein the antibody comprises a heavy chain having the amino acid sequence shown in SEQ ID NO: 111. In some embodiments, provided is an isolated monoclonal antibody that binds to human activated Protein C, wherein the antibody comprises a heavy chain having the amino acid sequence shown in SEQ ID NO: 113. In some embodiments, provided is an isolated monoclonal antibody that binds to human activated Protein C, wherein the antibody comprises a heavy chain having the amino acid sequence shown in SEQ ID NO: 115. In some embodiments, provided is an isolated monoclonal antibody that binds to human activated Protein C, wherein the antibody comprises a heavy chain having the amino acid sequence shown in SEQ ID NO: 117. In some embodiments, provided is an isolated monoclonal antibody that binds to human activated Protein C, wherein the antibody comprises a heavy chain having the amino acid sequence shown in SEQ ID NO: 119.

In some embodiments, provided is an isolated monoclonal antibody that binds to human activated Protein C, wherein the antibody comprises a light chain having the amino acid sequence shown in SEQ ID NO: 12, wherein the amino acid sequence comprises one or more amino acid modifications. In some embodiments, the modification of the light chain is a substitution, an insertion or a deletion. In some embodiments, the modifications are located in the CDRs of the light chain. In other embodiments, the modifications are located outside the CDRs of the light chain.

In some embodiments, the modification of the light chain of SEQ ID NO:12 is at a position selected from T25, D52, N53, N54, N55, D95, N98 or G99. The modification can be for example the one of the following substitutions: T25S, D52Y, D52F, D52L, D52G, N53C, N53K, N53G, N54S, N55K, D95G, N98S, G99H, G99L or G99F. Further, in some embodiments, the antibody may comprise two or more substitutions from T25S, D52Y, D52F, D52L, D52G, N53C, N53K, N53G, N54S, N55K, D95G, N98S, G99H, G99L or G99F.

In a further embodiment, provided is an isolated anti-aPC monoclonal antibody that binds to the human activated form of Protein C, wherein the antibody comprises a heavy chain having the amino acid sequence shown in SEQ ID NO: 22, wherein the amino acid sequence comprises one or more amino acid modifications. In some embodiments, the modification of the light chain is a substitution, an insertion or a deletion.

Epitopes

Also provided is an isolated monoclonal antibody that bind to an epitope of human activated Protein C, wherein the epitope comprises one or more of residues from the heavy chain of human aPC shown in SEQ ID NO:3.

In some embodiments, the epitope can include the active site of human aPC. In some embodiments, the active site can comprise amino acid residue 5195 of human aPC.

In some embodiments, the epitope can comprises one or more residues selected from D60, K96, S97, T98, T99, E170, V171, M172, 5173, M175, A190, 5195, W215, G216, E217, G218, and G218 of human activated Protein C shown in SEQ ID NO:3.

Also provided are antibodies which can compete with any of the antibodies described herein for binding to human activated Protein C. For example, such a competing antibody can bind to one or more epitopes described above.

Nucleic Acids, Vectors and Host Cells

Also provided are isolated nucleic acid molecules encoding any of the monoclonal antibodies described above.

Thus, provided is an isolated nucleic acid molecule encoding an antibody that binds to human activated Protein C.

In some embodiments, provided are isolated nucleic acid molecules encoding an antibody that binds to activated Protein C and inhibits anticoagulant activity but has minimal binding to unactivated Protein C, wherein the antibody comprises a heavy chain variable region comprising a nucleic acid sequence selected from the group consisting of SEQ ID Nos: 34-43.

In some embodiments, provided are isolated nucleic acid molecules encoding an antibody that binds to activated Protein C and inhibits anticoagulant activity but has minimal binding to unactivated Protein C, wherein the antibody comprises a light chain variable region comprising a nucleic acid sequence selected from the group consisting of SEQ ID Nos: 24-33.

In some embodiments, provided are isolated nucleic acid molecules encoding an antibody that binds to activated Protein C and inhibits anticoagulant activity but has minimal binding to unactivated Protein C, wherein the antibody comprises a heavy chain variable region comprising an amino acid sequence selected from the group consisting of SEQ ID Nos: 14-23.

In some embodiments, provided are isolated nucleic acid molecules encoding an antibody that binds to activated Protein C and inhibits anticoagulant activity but has minimal binding to unactivated Protein C, wherein the antibody comprises a light chain variable region comprising an amino acid sequence selected from the group consisting of SEQ ID Nos: 4-13.

In another embodiment, provided are isolated nucleic acid molecules encoding an antibody that binds to activated Protein C and inhibits anticoagulant activity but has minimal binding to unactivated Protein C, wherein the antibody comprises a heavy chain variable region comprising an amino acid sequence selected from the group consisting of SEQ ID Nos: 14-23 or a light chain variable region comprising an amino acid sequence selected from the group consisting of SEQ ID Nos: 4-13, and one or more amino acid modifications in the heavy chain variable region or light chain variable region.

Further, also provided are vectors comprising the isolated nucleic acid molecules encoding any of the monoclonal antibodies described above and host cells comprising such vectors.

Methods of Preparing Antibodies to aPC

The monoclonal antibody can be produced recombinantly by expressing a nucleotide sequence encoding the variable regions of the monoclonal antibody according to one of the present embodiments in a host cell. With the aid of an expression vector, a nucleic acid containing the nucleotide sequence can be transfected and expressed in a host cell suitable for the production. Accordingly, also provided is a method for producing a monoclonal antibody that binds with human aPC comprising:

(a) transfecting a nucleic acid molecule encoding a monoclonal antibody into a host cell,

(b) culturing the host cell so to express the monoclonal antibody in the host cell, and optionally isolating and purifying the produced monoclonal antibody, wherein the nucleic acid molecule comprises a nucleotide sequence encoding a monoclonal antibody.

In one example, to express the antibodies, or antibody fragments thereof, DNAs encoding partial or full-length light and heavy chains obtained by standard molecular biology techniques are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain encoding genes, the recombinant expression vectors carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences can depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences can be used, such as the ubiquitin promoter or β-globin promoter.

In addition to the antibody chain genes and regulatory sequences, the recombinant expression vectors can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Examples of selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies in either prokaryotic or eukaryotic host cells, expression of antibodies in eukaryotic cells, including mammalian host cells, is typical because such eukaryotic cells, and in particular mammalian cells, are more likely than prokaryotic cells to assemble and secrete a properly folded and immunologically active antibody.

Examples of mammalian host cells for expressing the recombinant antibodies include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells, HKB11 cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods, such as ultrafiltration, size exclusion chromatography, ion exchange chromatography and centrifugation.

Use of Partial Antibody Sequences to Express Intact Antibodies

Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998, Nature 332:323-327; Jones, P. et al., 1986, Nature 321:522-525; and Queen, C. et al., 1989, Proc. Natl. Acad. Sci. U.S.A. 86:10029-10033). Such framework sequences can be obtained from public DNA databases that include germline antibody gene sequences. These germline sequences will differ from mature antibody gene sequences because they will not include completely assembled variable genes, which are formed by V(D)J joining during B cell maturation. It is not necessary to obtain the entire DNA sequence of a particular antibody in order to recreate an intact recombinant antibody having binding properties similar to those of the original antibody (see WO 99/45962). Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. For this reason, it is necessary to use the corresponding germline leader sequence for expression constructs. To add missing sequences, cloned cDNA sequences can be combined with synthetic oligonucleotides by ligation or PCR amplification. Alternatively, the entire variable region can be synthesized as a set of short, overlapping, oligonucleotides and combined by PCR amplification to create an entirely synthetic variable region clone. This process has certain advantages such as elimination or inclusion or particular restriction sites, or optimization of particular codons.

The nucleotide sequences of heavy and light chain transcripts are used to design an overlapping set of synthetic oligonucleotides to create synthetic V sequences with identical amino acid coding capacities as the natural sequences. The synthetic heavy and light chain sequences can differ from the natural sequences. For example: strings of repeated nucleotide bases are interrupted to facilitate oligonucleotide synthesis and PCR amplification; optimal translation initiation sites are incorporated according to Kozak's rules (Kozak, 1991, J. Biol. Chem. 266:19867-19870); and restriction sites are engineered upstream or downstream of the translation initiation sites.

For both the heavy and light chain variable regions, the optimized coding, and corresponding non-coding, strand sequences are broken down into 30-50 nucleotide sections at approximately the midpoint of the corresponding non-coding oligonucleotide. Thus, for each chain, the oligonucleotides can be assembled into overlapping double stranded sets that span segments of 150-400 nucleotides. The pools are then used as templates to produce PCR amplification products of 150-400 nucleotides. Typically, a single variable region oligonucleotide set will be broken down into two pools which are separately amplified to generate two overlapping PCR products. These overlapping products are then combined by PCR amplification to form the complete variable region. It can also be desirable to include an overlapping fragment of the heavy or light chain constant region in the PCR amplification to generate fragments that can easily be cloned into the expression vector constructs.

The reconstructed heavy and light chain variable regions are then combined with cloned promoter, translation initiation, constant region, 3′ untranslated, polyadenylation, and transcription termination sequences to form expression vector constructs. The heavy and light chain expression constructs can be combined into a single vector, co-transfected, serially transfected, or separately transfected into host cells which are then fused to form a host cell expressing both chains.

Thus, in another aspect, the structural features of a human anti-aPC antibody are used to create structurally related human anti-aPC antibodies that retain the function of binding to aPC. More specifically, one or more CDRs of the specifically identified heavy and light chain regions of the monoclonal antibodies can be combined recombinantly with known human framework regions and CDRs to create additional, recombinantly-engineered, human anti-aPC antibodies.

Pharmaceutical Compositions

Also provided are pharmaceutical compositions comprising therapeutically effective amounts of anti-aPC monoclonal antibody and a pharmaceutically acceptable carrier. “Pharmaceutically acceptable carrier” is a substance that can be added to the active ingredient to help formulate or stabilize the preparation and causes no significant adverse toxicological effects to the patient. Examples of such carriers are well known to those skilled in the art and include water, sugars such as maltose or sucrose, albumin, salts such as sodium chloride, etc. Other carriers are described for example in Remington's Pharmaceutical Sciences by E. W. Martin. Such compositions will contain a therapeutically effective amount of at least one anti-TFPI monoclonal antibody.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. The composition is in some embodiments formulated for parenteral injection. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In some cases, it will include isotonic agents, for example, sugars, polyalcohols such as mannitol, sorbitol, or sodium chloride in the composition.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof

Pharmaceutical Uses

The monoclonal antibody can be used for therapeutic purposes for treating genetic and acquired deficiencies or defects in coagulation. For example, the monoclonal antibodies in the embodiments described above can be used to block the interaction of aPC with its substrate, which can include Factor Va or Factor VIIIa.

The monoclonal antibodies have therapeutic use in the treatment of disorders of hemostasis such as thrombocytopenia, platelet disorders and bleeding disorders (e.g., hemophilia A, hemophilia B and hemophilia C). Such disorders can be treated by administering a therapeutically effective amount of the anti-aPC monoclonal antibody to a patient in need thereof. The monoclonal antibodies also have therapeutic use in the treatment of uncontrolled bleeds in indications such as trauma and hemorrhagic stroke. Thus, also provided is a method for shortening the bleeding time comprising administering a therapeutically effective amount of an anti-aPC monoclonal antibody to a patient in need thereof.

In another embodiment, the anti-aPC antibody can be useful as an antidote for aPC-treated patients, including for example wherein aPC is used for the treatment of sepsis or bleeding disorder.

The antibodies can be used as monotherapy or in combination with other therapies to address a hemostatic disorder. For example, co-administration of one or more antibodies with a clotting factor such as factor VIIa, factor VIII or factor IX is believed useful for treating hemophilia. In one embodiment, provided is a method for treating genetic and acquired deficiencies or defects in coagulation comprising administering (a) a first amount of a monoclonal antibody that binds to human tissue factor pathway inhibitor and (b) a second amount of factor VIII or factor IX, wherein said first and second amounts together are effective for treating said deficiencies or defects. In another embodiment, provided is a method for treating genetic and acquired deficiencies or defects in coagulation comprising administering (a) a first amount of a monoclonal antibody that binds to human tissue factor pathway inhibitor and (b) a second amount of factor VIII or factor IX, wherein said first and second amounts together are effective for treating said deficiencies or defects, and further wherein factor VII is not coadministered. Also included is a pharmaceutical composition comprising a therapeutically effective amount of the combination of a monoclonal antibody and factor VIII or factor IX, wherein the composition does not contain factor VII. “Factor VII” includes factor VII and factor VIIa. These combination therapies are likely to reduce the necessary infusion frequency of the clotting factor. By co-administration or combination therapy is meant administration of the two therapeutic drugs each formulated separately or formulated together in one composition, and, when formulated separately, administered either at approximately the same time or at different times, but over the same therapeutic period.

In some embodiments, one or more antibodies described herein can be used in combination to address a hemostatic disorder. For example, co-administration of two or more of the antibodies described herein is believed useful for treating hemophilia or other hemostatic disorder.

The pharmaceutical compositions can be parenterally administered to subjects suffering from hemophilia A or B at a dosage and frequency that can vary with the severity of the bleeding episode or, in the case of prophylactic therapy, can vary with the severity of the patient's clotting deficiency.

The compositions can be administered to patients in need as a bolus or by continuous infusion. For example, a bolus administration of an inventive antibody present as a Fab fragment can be in an amount of from 0.0025 to 100 mg/kg body weight, 0.025 to 0.25 mg/kg, 0.010 to 0.10 mg/kg or 0.10-0.50 mg/kg. For continuous infusion, an inventive antibody present as an Fab fragment can be administered at 0.001 to 100 mg/kg body weight/minute, 0.0125 to 1.25 mg/kg/min., 0.010 to 0.75 mg/kg/min., 0.010 to 1.0 mg/kg/min. or 0.10-0.50 mg/kg/min. for a period of 1-24 hours, 1-12 hours, 2-12 hours, 6-12 hours, 2-8 hours, or 1-2 hours. For administration of an inventive antibody present as a full-length antibody (with full constant regions), dosage amounts can be about 1-10 mg/kg body weight, 2-8 mg/kg, or 5-6 mg/kg. Such full-length antibodies would typically be administered by infusion extending for a period of thirty minutes to three hours. The frequency of the administration would depend upon the severity of the condition. Frequency could range from three times per week to once every two weeks to six months.

Additionally, the compositions can be administered to patients via subcutaneous injection. For example, a dose of 10 to 100 mg anti-aPC antibody can be administered to patients via subcutaneous injection weekly, biweekly or monthly.

As used herein, “therapeutically effective amount” means an amount of an anti-aPC monoclonal antibody or of a combination of such antibody and factor VIII or factor IX that is needed to effectively increase the clotting time in vivo or otherwise cause a measurable benefit in vivo to a patient in need. The precise amount will depend upon numerous factors, including, but not limited to the components and physical characteristics of the therapeutic composition, intended patient population, individual patient considerations, and the like, and can readily be determined by one skilled in the art.

### EXAMPLES

Aspects of the present disclosure may be further understood in light of the following examples, which should not be construed as limiting the scope of the present teachings in any way.

### Example 1

### Materials and Methods

Screening of Human aPC-Specific Binders

Preparation of Master Plates: Master plates were produced by picking 1880 clones per panning strategy into 384 well plates (ThermoFisher Scientific, Weltham, Mass. USA) containing growth media (2XYT/1% glucose/100 μg/ml Carbenicillin) using the Qpix2 (Genetix, Boston, Mass. USA) colony picker. Plates were grown overnight at 37° C. with shaking.

Production of Expression plates: Using the Evolution P3 liquid handler (Perkin Elmer, Waltham, Mass., USA) 5 μl of media from the master plates were transferred to 384 well plates containing expression media (2XYT/0.1% glucose/100 ug/ml Carb) and incubated at 30° C. When the cultures reach an OD 600 of 0.5, IPTG is added at a final concentration of 0.5 mM. Plates are then returned to 30° C. for overnight growth.

Primary ELISA: Maxisorp 384 well plates (ThermoFisher Scientific, Rochester, N.Y. USA) were coated with recombinant human aPC or human PC (Mol. Innovation) at 1 μg/ml in DPBS with Ca/Mg and incubated overnight at 4° C. Coated ELISA plates were washed three times with DPBST (PBS+0.05% TWEEN) and blocked with MDPBST (PBS+0.05% TWEEN+5% Milk) for 1 hr at RT. Blocked plates were aspirated and 15 μl expression media and 30 μl MDPBST were transferred to each well. ELISA plates were incubated at room temperature for 1 hr, followed by 5 times of wash with DPBST. Anti-hFab-HRP (Jackson ImmunoResearch, 1:10,000 dilution in DPBST) was added to each well and incubated for 1 hr at room temperature. Plates were then washed 5 times with DPBST. Amplex Red (Invitrogen) substrate was added and plates were read at an excitation of 485 nm and emission of 595 nm.

Confirming ELISA: Using the Qpix2 colony picker, putative positive clones were rearrayed from the master plates into 96 deep-well plates (Qiagen) containing 1 ml growth media and grown overnight at 37° C. Expression plates were inoculated from the master plates and induced with IPTG at 0.5 mM final concentration when the cultures reached an OD600 of 0.5. ELISA was then performed on the expression media as outlined above.

Library Selections with Biotinylated aPC (in-Solution Panning)

Two methods were carried out: depletion of PC binders and non-depletion for total PC and aPC binders. Dynabeads M280 Streptavidin was coupled to 100 nM biotin-TF (tissue factor, for non-depletion) or 100 nM biotin-PC (depletion) and captured by magnetic device. 1-7.5×1012 cfu Fab library phage, pre-blocked with DPBS/3% BSA/0.05% TWEEN 20, was incubated with biotin-TF or biotin-PC coupled Strepavidin beads on a rotator at room temperature for 2 hours. The biotin-TF (non-depletion) or biotin-PC (depletion)/Streptavidin beads were captured and discarded. The resulting phage supernatants were incubated with 100 nM (first round), 50 nM (second round) or 10 nM (third round) biotin-aPC in 1 ml DPBS/3% BSA/0.05% TWEEN 20/1 mM CaCl2 for 2 hours at RT or 40° C. overnight. 100 ul of Streptavidin-coupled magnetic beads were added to the phage-aPC solution and incubated for 30 minutes at room temperature. The phage-aPC complex beads was captured on magnetic device and washed with various times of DPBS with 3% BSA or 0.05% TWEEN 20 depending upon the panning rounds. The bound phage was eluted with 1 mg/ml trypsin and neutralized with aprotinin. The eluted phage was then used to infect 10 ml exponentially growing E. coli HB101F′ and amplified for the next round of selection. The phage stock was also analyzed in a CFU titration (panning output).

Library Selections with Immobilized aPC (Solid-Phase Panning)

Five wells of Maxi-sorp 96-well plate was coated with 400 ng/well recombinant aPC in DPBS at 4° C. overnight. The same as in-solution panning, the phage library was pre-treated with biotin-TF for non-depletion or biotin-PC for depletion. The resulting phage then was added to the aPC coated wells and incubated on a shaker for 1-2 hours at room temperature. Unbound phage was washed away by washing with various times of DPBS with 3% BSA or 0.05% TWEEN 20 depending upon the panning rounds. The bound phage was eluted with 1 mg/ml trypsin and neutralized with aprotinin. The eluted phage was then used to infect 10 ml exponentially growing E. coli HB101F′ and amplified for the next round of selection. The phage stock was also analyzed in a CFU titration (panning output).

Amplification of Selected Phage Pools: Eluted Phage Stocks were Amplified in HB101F′ Using Helper Phage M13K07 for Selection Round 2, 3 and 4.

A volume of 10 ml of exponentially growing HB101F′ was infected with eluted phage from each round of selection and incubated at 37° C. for 45 minutes, 50 rpm. The bacteria were then resuspended in 2×YT medium and spread on two 15 cm agar plates containing 100 μg/ml carbocinin, 15 μg/ml tetracycline and 1% glucose followed by overnight incubation at 30° C. The lawn of bacteria from the plates were collected with total of 8 ml 2×YT/carb/tet.

About 10 μl of cells were resuspended in 10 ml of 2×YT/carb/tet (0D600 is around 0.1-0.2) and incubated at 37° C. until OD600 reached 0.5-0.7. 5×1010 cfu of M13K07 helper phage was added to the cells and incubated for 45 minutes at 37° C. The infected cells were then resuspended in 15 ml of fresh 2×YT/carb/kanamycin (50 μg/ml)/tet and shaking overnight at 30° C. to produce phage. The phage supernatant was collected by centrifugation and filtration through 0.45 μm filter. 900 μl of the supernatant was used for next round of selection.

### DNA Sequencing Analysis of aPC Antibodies

Plasmid was prepared using standard molecular biology techniques. The following primers were used for DNA sequencing of selected antibody clones.

a) Primer A:
5′ GAAACAGCTATGAAATACCTATTGC 3′
b) Primer B:
5′ GCCTGAGCAGTGGAAGTCC 3′
c) Primer C:
5′ TAGGTATTTCATTATGACTGTCTC 3′
d) Primer D:
5′ CCCAGTCACGACGTTGTAAAACG 3′

Purification of Protein C from Plasma.

One liter of dog or rabbit plasma was purchased as 20×50 ml frozen stocks with heparin included as anticoagulant (Bioreclamation, Inc., Westbury, N.Y.). The purification method was described by Esmon's lab (12) with modifications. Plasma was thawed at 4 C, and diluted 1:1 with 0.02M Tris-HCl, pH7.5, heparin 1U/ml final, benzamidine HCl 10 mM final, at RT before loading onto a Q-Sepharose column for capturing protein C and other vitamin K-dependent proteins. The column was washed with buffered 0.15M NaCl, and protein C was eluted with buffered 0.5M NaCl. Eluents were recalcified with 10 mM Ca++ and 100 U/ml heparin and then loaded onto HCP4-Affigel-10 affinity column. The column was washed with Ca-containing buffer and eluted with EDTA-containing buffer. Purified PC was dialyzed overnight into PBS buffer, flash frozen and stored at −80 as 0.5 ml aliquots. The purification yield was 1.75 mg from one liter dog plasma. The Purified PC had 98% purity as determined by SDS-PAGE and analytical SEC.

### Fab Expression and Purification

For Fab expression, 5·1 sFab E. coli glycerol stock was inoculated into 1 ml growth media (LB, 1% glucose, 100·g/ml ampicillin), and the culture grew at 37° C. overnight with shaking at 250 rpm. The overnight culture 500·1 was then inoculated into 10 ml prewarmed (37° C.) induction media (LB, 0.1% glucose, 100·g/ml ampicillin) and grew at 37° C. to OD500 0.6-0.7 at 250 rpm. IPTG was added to the culture to 0.5 mM final concentration for Fab expression, and the culture grew overnight at 30 C with shaking at 250 rpm. Next day, the overnight culture was centrifuged at 3,000 g for 15 min at 4° C. to separate the media from cells. Both supernate and pellet were saved for Fab purification. Fab expression in both supernate and pellet can be confirmed by western blot analysis using anti-His antibody.

For Fab purification, Protein A column (MabSure) was used as recommended by the BioInvent protocol. Supernate was filtered through a 0.45 um filter to remove debris and mixed with a tablet of complete protease inhibitors (Roche 11873580001) before loading onto a buffer-equilibrated protein A column. Fab was eluted with pH 2-3 buffer then buffer-exchanged to PBS, pH 7.0. In order to liberate Fab from cell pellets, 1 ml lysis buffer was added to pellet. The mixture was incubated for 1 h for lysis at 4° C. on a rocking platform then centrifuged at 3,000 g for 30 min at 4° C. Clear supernate was transferred to a new tube and loaded onto Protein A column. Lysis buffer contains freshly prepared 1 mg/ml lysozyme (Sigma L-6876) in cold sucrose solution (20% sucrose (w/v), 30 mM TRIS-HCL, 1 mM EDTA, pH 8.0), 2.5 U/ml benzonase (Sigma E1014) (25 KU/ml, stock solution 1/10.000), and 1 tablet of complete protease inhibitors (Roche 11873580001). Purity of the purified Fab was confirmed by SDS-PAGE and analytical size-exclusion chromatography (SEC). Endotoxin levels were also monitored.

### Western Blot Analysis of PC and aPC.

Purified protein (100 ng/lane) was mixed with 4×SDS-PAGE loading dye with DTT (reducing) or without DTT (non-reducing), heated at 95° C. for 5 min then loaded onto 4-12% NuPAGE gels. Proteins were transferred to nitrocellulose membranes by i-Blot (Life technologies, Carlsbad, Calif.). Probing steps were done with SNAP-id (Millipore). After blocking with 5% milk/PBS for 10 min, the membranes were incubated with various reagents (e.g. Streptavidin-HRP for detection of biotinylated aPC, the mouse anti-human PC monoclonal antibody HCP-4 and anti-PC goat polyclonal antibody for detection of dog aPC). The probing was followed by incubation with HRP secondary antibody for 10 minutes at room temperature. After washing the blots with PBS with 0.1% TWEEN-20, the signal from HRP was detected using a chemiluminescent substrate (ECL) (Pierce, Rockford, Ill.) and exposure to x-ray film.

### Fab ELISA

Antigen proteins (human PC, human PC, mouse APC, dog APC) were coated to an ELISA plate at 100 ng/100 ul/well in PBS/Ca buffer (Life technologies) overnight at 4° C. The next day, the plate was washed 3× and blocked with 5% PBS/Ca/BSA/Tween20 for 1 h at RT. Soluble Fab was added to each well and incubated for 1 h at RT. After adding the anti-human lambda-antibody-HRP as detection antibody, the plate was incubated at room temperature for 1 hr, washed extensively and then developed using Amplex Red substrate as described by the kit manufacturer. The signal was measured as RFU using a fluorescent plate reader (SpectraMax 340pc, Molecular Devices, Sunnyvale, Calif.). The standard curve was fitted to a four-parameter model, and the values of the unknowns were extrapolated from the curve.

### Example 2

### Panning of aPC Antibody from Library

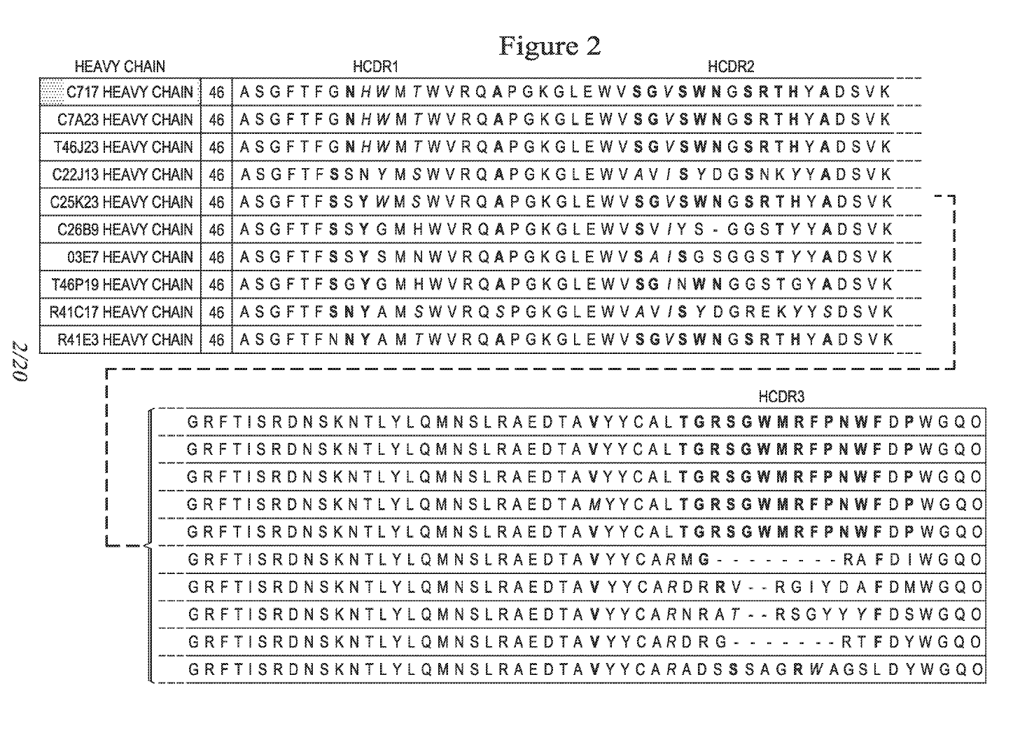

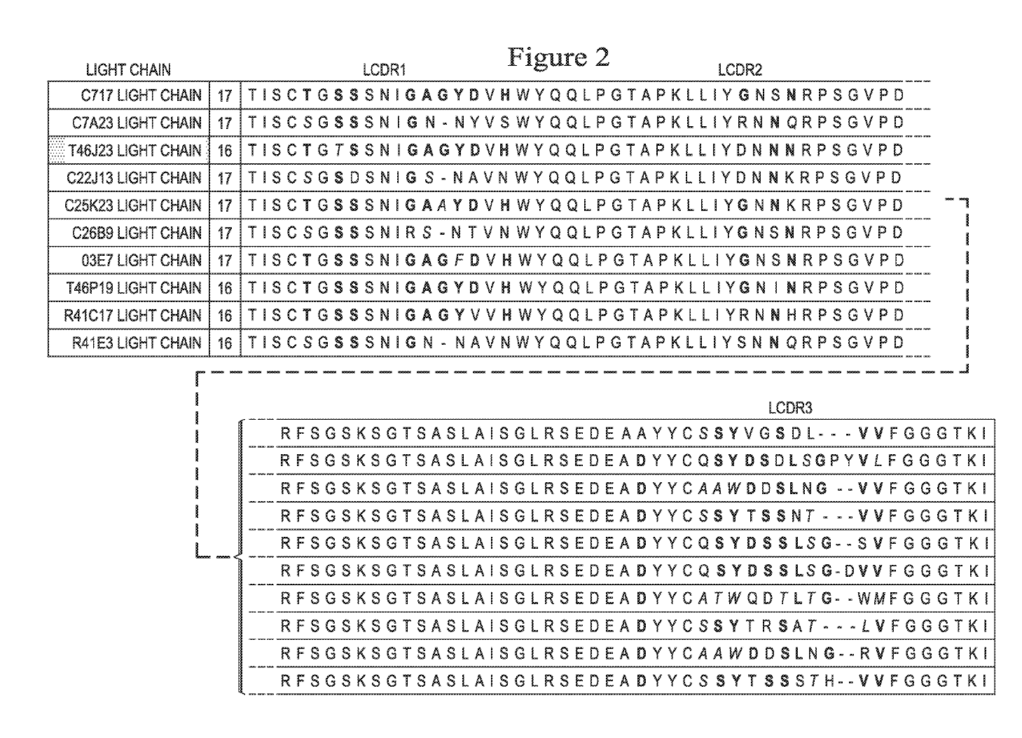

Panning and screening of a fully human Fab antibody library against human activated Protein C was performed using the methods as described in Example 1. DNA sequencing was performed on the positive antibody clones resulting in 10 unique antibody sequences. An alignment of the heavy chain and light chains of the antibodies is shown in FIG. 2. Identical heavy chain CDR3 sequences are found in 5 Fabs (C7I7, C7A23, T46J23, C22J13, C25K23).

The purified Fabs were characterized by a panel of functional assays to assess: a) their binding specificity (aPC vs. PC); binding affinity (by ELISA and Biacore); and species cross-reactivity (ie. Binding to aPCs of different species origins including human, dog and mouse). Rabbit aPC was also used later for IgG format; b) their binding selectivity against other vitamin K-dependent coagulation factors (e.g. FIIa, FVIIa, FIXa, FXa); c) their potency of inhibiting aPC's anti-coagulant activity in the plasma clotting assay aPTT; and d) their effect on aPC's protease enzymatic activity in buffer using amidolytic activity assay (on a small peptide substrate) and FVa inactivation assay (on the protein substrate FVa).

### Example 3

### Binding Affinity of aPC-Specific Antibodies and Cross-Species Reactivity

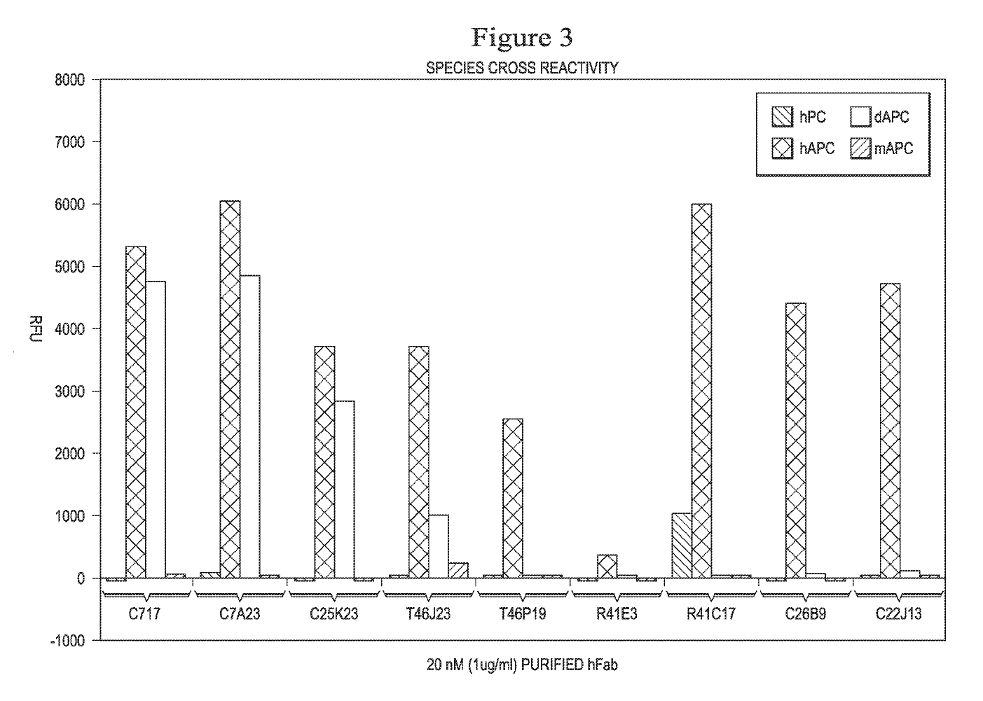

Antigen-binding activities of these purified anti-aPC Fabs were determined by direct ELISA as shown in FIG. 3. Antigens were coated directly on ELISA plates. Coating antigens included human PC (plasma-derived), human aPC (recombinant), dog aPC (plasma-derived), and mouse aPC (recombinant) at 100 ng/well in PBS/Ca buffer. After blocking the plate with 5% milk/PBS and washing the plate with PBS-Tween20, soluble Fabs (1 ug/ml, 20 nM) were added to the plate and incubated for 1 h at RT with shaking. Fab binding was detected with anti-human Fab (lambda) antibody-HRP and Amplex red as substrate. ELISA data showed that all Fabs specifically bind to human aPC but not to human PC. One Fab, R41C17, showed minimal binding to human PC. In contrast, R41C17 binds to both human APC and human PC. Also shown in FIG. 3 is cross-species reactivity of Fabs by ELISA. Among 8 aPC-specific binders, 4 of them (C7I7, C7A23, C25K23, T46J23) also showed cross-reactivity with dog aPC. Further, one Fab, T46J23, showed some binding mouse aPC.

Shown in Table 3 is the EC₅₀ as measured by ELISA of anti-aPC antibodies to human aPC and dog aPC.

TABLE 3
ELISA analysis of anti-aPC Fabs

| | Fab/EC₅₀ (nM) | | | | | | | | |
|---|---|---|---|---|---|---|---|---|---|
| | C7A23 | C7I7 | C22J13 | C25K23 | C26B9 | R41C17 | R41E3 | T46J23 | T46P19 |
| Human | 2.4 | 6.3 | 10.6 | 4.3 | 10.3 | 8.6 | 10.4 | 6.7 | 10.3 |
| aPC | | | | | | | | | |
| Dog | 6 | 3.9 | | 8.2 | | | | 16.6 | |
| aPC | | | | | | | | | |

The affinity of the anti-aPC Fabs was determined by Biacore and is shown in Table 4.

TABLE 4
ELISA analysis of anti-aPC Fabs

| | Fab/KD (nM) | | | | | | | | |
|---|---|---|---|---|---|---|---|---|---|
| | C7A23 | C7I7 | C22J13 | C25K23 | C26B9 | R41C17 | R41E3 | T46J23 | T46P19 |
| Human | 1.9 | 1.3 | 16 | 2 | 7.9 | 4.8 | 11 | 4.1 | 7.2 |
| aPC | | | | | | | | | |
| Dog | 1.3 | 1.3 | 300 | 5.2 | | | | 21 | |
| aPC | | | | | | | | | |

### Example 4

### Binding Selectivity of Anti-aPC Fabs

To determine the binding selectivity of these fabs, their binding activities to the proenzyme human PC, to thrombin (FIIa), and to the activated Factor II (FIIa, thrombin), Factor VII (FVIIa), Factor IX (FIXa), and Factor X (FXa) were also assessed by ELISA. Briefly, an ELISA plate was coated with human aPC at 1 ug/ml, mouse PC at 10 ug/ml, dog PC at 10 ug/ml, other coagulation factors (FIIa, FVIIa, FIXa, FXa) at 5-10 ug/ml. Anti-aPC Fabs were added to the wells at 20 nM (1 ug/ml). Bound Fabs were detected by the secondary antibody (anti-human Fab-HRP) followed by HRP substrate AmplexRed. As positive control, a control antibody specific for each antigen was used to demonstrate that coating antigen is present.

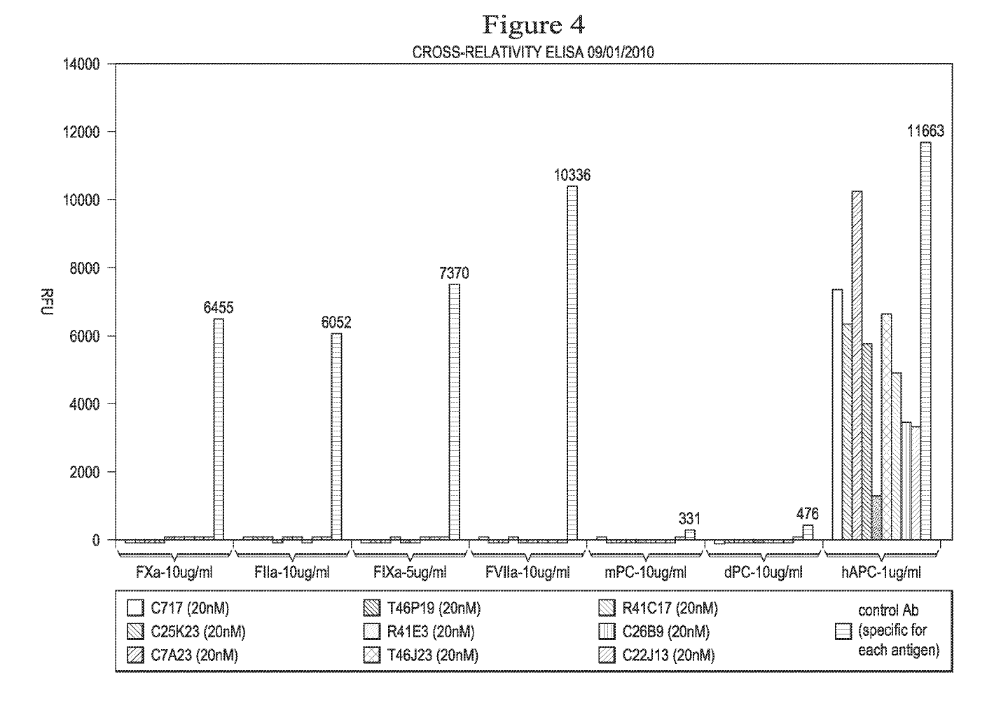

As shown in FIG. 4, up to a concentration of 20 nM, none of the Fabs showed binding to the factors IIa, VIIa, IXa, or Xa. Binding to the proenzyme mouse PC or dog PC was also not detectable.

### Example 5

### Anti-aPC Fabs Inhibit aPC and Induce Clot Formation in Normal Human Plasma

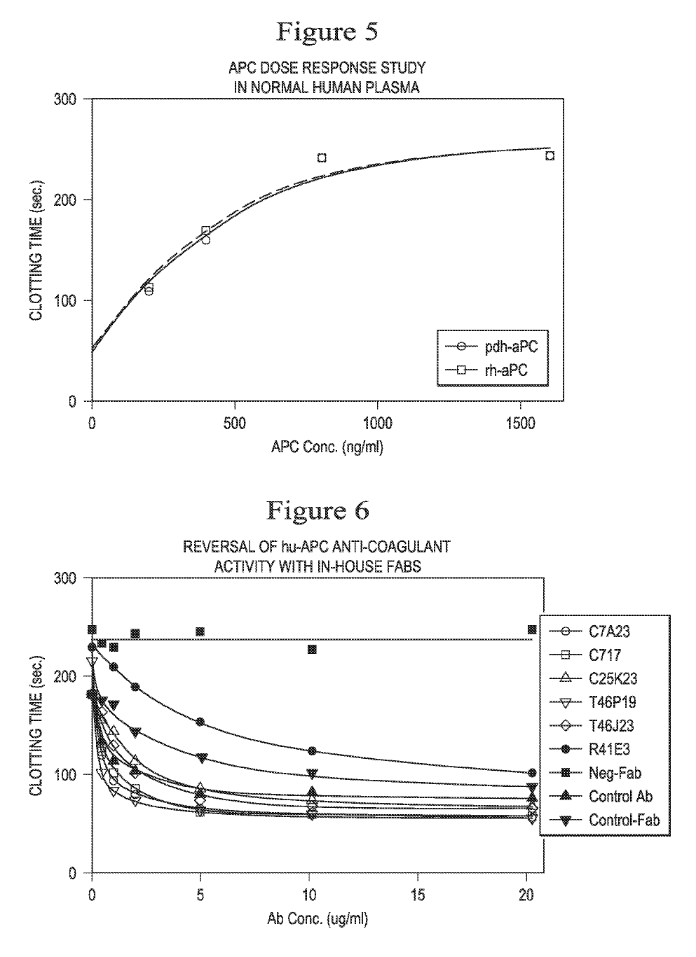

Human aPC is a potent anti-coagulant, and this function can be easily demonstrated by the plasma clotting assay (aPTT) as shown in FIG. 5. In aPTT assays, 50% normal human pooled plasma formed clots in 52 seconds upon adding CaCls (initiator) to the mixture of plasma and phospholipids. Preincubation of human aPC at 100, 200, 400, 800, or 1600 ng/ml with plasma prolonged the clotting time in a dose-dependent manner. As shown in FIG. 5, nearly identical potency was obtained for plasma-derived aPC and the recombinant aPC. Since the maximal setting of clotting time for the Stago instrument was 240 seconds, the anti-coagulant activity of human aPC in this functional assay reached its maximum at 800 ng/ml of aPC.

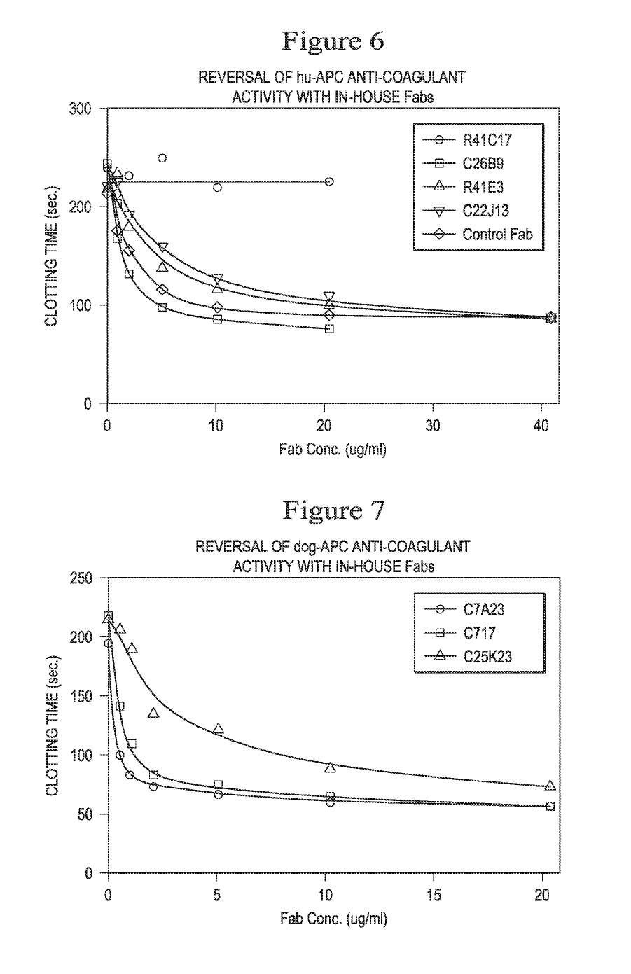

To evaluate potential inhibitory effects of anti-aPC Fabs on the anti-coagulant activity of aPC, 400 ng/ml aPC was used in aPTT assays for a good assay range (FIG. 6). The plasma clotting time extended from 52 seconds to 180 seconds due to the anti-coagulant activity of administered aPC. Incubation of a tool mouse anti-human APC antibody (control) or its Fab (control-Fab) or Fab C7A23 at 0, 0.5, 1, 2, 5, 10, or 20 ug/ml with aPC (i.e. 1.5× to 60× fold excess of Fab over aPC) reduced the clotting time in a dose-dependent manner. Fab C7A23 was 4-5-fold more potent than contrl-Fab in reversing the anti-coagulant activity of human aPC. In contrast, the negative control Fab (human Fab lambda) had no effect on the clotting time. In FIG. 6, the full-length control antibody (bivalent) was 10-fold more potent than control-Fab (monovalent) in aPTT assay. This result was consistent with their EC50 values [control (0.56 nM) vs. control-Fab (6.56 nM)] in direct ELISA for aPC binding (data not shown). Thus, suggesting a more potent molecule when the anti-aPC Fabs are converted into IgG format. The aPTT results suggest that the anti-aPC Fabs significantly inhibited the anti-coagulant activity of aPC and shortened the clotting time. All the tested Fabs were evaluated in plasma clotting assay aPTT in comparison with the control-Fab (FIG. 6). In the upper graph of FIG. 6, a non-specific human Fab was used as negative control, and it did not affect clotting time as expected. Positive controls (control and control-Fab) shortened the clotting time in a dose-dependent manner.

Fabs C7A23, C7I7, C25K23, T46J23, and T46P19 at 5 ug/ml (15-fold molar excess over spiked-in aPC) caused 80-93% inhibition of human aPC activity and enhanced clot formation. They were clearly more potent than control-Fab. In contrast, Fab R41E3 only produced 30-40% inhibition of aPC activity under identical conditions. The weak activity of R41E3 in aPTT likely resulted from its lower affinity of aPC binding as determined by ELISA and Biacore. An increase in the R41E3 Fab concentration to 40 ug/ml (100-fold molar excess over aPC) indeed caused 80% inhibition of human aPC as shown in lower graph of FIG. 6. Likewise, a high dose (40 ug/ml) of C22J13 Fab produced 80% inhibition of human aPC. Fab C26B9 was more potent than control-Fab in this assay. In the lower graph, Fab R41C17 had no effect on aPC activity, because it binds both PC and aPC and there are over 1000-fold more abundant PC than aPC in human plasma. This data also indicates that Fab R41C17 has a different binding epitope from the other Fabs.

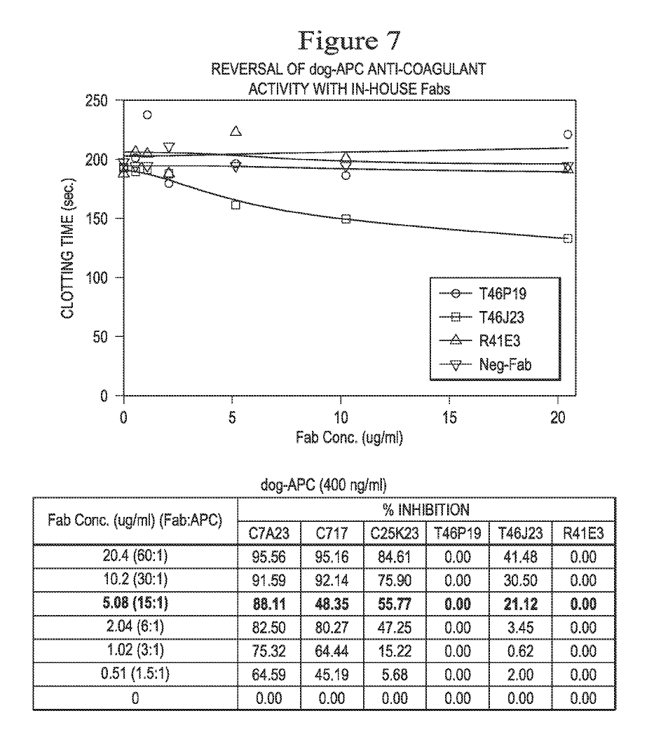

As indicated by species aPC ELISA data, 4 Fabs (C7A23, C7I7, C25K23, T46J23) also bind to dog aPC at nanomolar affinity, these Fabs were evaluated by aPTT using dog aPC spiked into 50% pooled human normal plasma as shown in FIG. 7. Dog aPC exhibited identical anti-coagulant activity as human aPC by aPTT (data not shown). The dog aPC at 300 ng/ml increased the clotting time from 47 seconds to 117 seconds. Incubation of the control antibody or control-Fab at 0, 0.5, 1, 2, 5, 10, or 20 ug/ml with dog aPC did not affect the clotting time because they do not cross-react with dog aPC by ELISA. However, the Fab C7A23 significantly reduced clotting time in a dose-dependent manner and inhibited the dog aPC activity up to 80% at 5 ug/ml or 85% at 20 ug/ml. Moreover, C7A23 showed comparable potency in blocking human aPC and dog aPC in aPTT assays. Fabs C7A23, C717, C25K23 clearly inhibited dog aPC activity in a dose-dependent manner. At 20 ug/ml Fab concentration, these 3 Fabs cause 80-90% inhibition of aPC and shorten the clotting time. Fab T46J23 gave only 40% inhibition at high dose, in consistent with its weaker binding to dog aPC (KD=22 nM) than C7A23, C7I7, C25K23 (KD=1-5 nM) by ELISA and Biacore. In contrast, Fabs T46P19 and R41E3 had no effect on dog aPC in APTT as expected since they could not bind to dog-aPC by ELISA.

### Example 6

### Effect of Anti-aPC Fabs on Enzymatic Activity of aPC

Activated Protein C is a serine protease. Its catalytic activity can be measured by two methods: a) amidolytic activity assay using a small peptide substrate, and b) FVa degradation assay using a physiological protein substrate FVa.

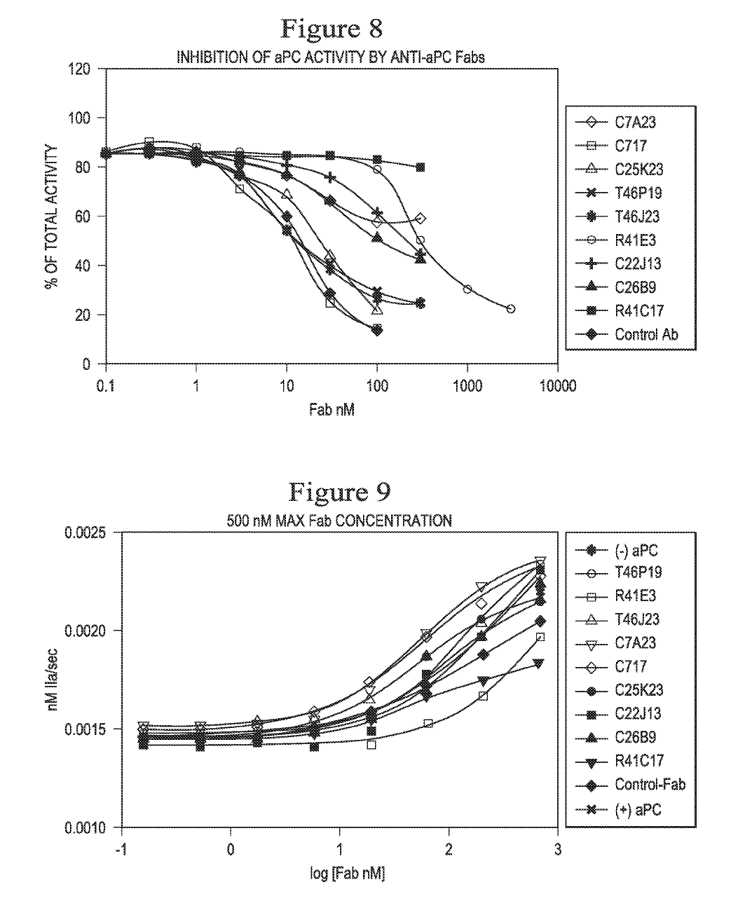

Amidolytic activity of human aPC was investigated by using a chromogenic peptide substrate of aPC in buffer. Purified aPC protein at 10 nM was incubated with the chromogenic substrate SPECTROZYME Pca (Lys-Pro-Arg-pNA, MW 773.9 Da) at 1 mM for 30 min. The conversion of substrate to colorimetric product (ie. Enzyme activity of aPC) was monitored by kinetically reading OD450 every 5 minutes. A standard curve was generated with recombinant human aPC. To test the effect of anti-aPC Fabs on aPC's amidolytic activity (FIG. 8), purified aPC protein (20 nM) was first preincubated with an equal volume of anti-aPC Fab (1-1000 nM) at RT for 20 min before the chromogenic substrate SPECTROZYME Pca was added to the reaction mixture up to 1 mM. The amidolytic activity of human aPC at a final concentration of 10 nM was measured in the presence of Fabs. Hydrolysis rates at a final substrate concentration of 1 mM were partially inhibited in the presence of the Fabs, reaching a maximum reduction of 80%. All Fabs except R41C17 inhibited aPC in a dose-dependent manner. IC50 correlated with EC50 in ELISA binding assay, as high-affinity binders (C7I7, C7A23, T46P19, T46J23, C25K23) showed much faster inhibition in this assay than the rest of weaker binders (R41E3, C22J13, C26B9). However, increasing concentration of Fabs for weaker binders also produced maximal inhibition. For example, R41E3 at 3,000 nM produced about 80% inhibition of aPC activity, and the same extent of inhibition was achieved by high affinity binders at 100 nM. Thus, most binders interacted with the active site of aPC causing the inhibition of its amidolytic activity. Interestingly, the control antibody caused partial inhibition of aPC (40%) and reached a plateau at concentrations greater than 100 nM. No inhibitory effect was observed when using increasing concentrations of R41C17 Fab. Since its binding affinity for human aPC is comparable to high affinity binders with a KD value of 4.8 nM by Biacore, these data indicate that R41C17 has a binding epitope far away from the enzymatic active site of aPC.

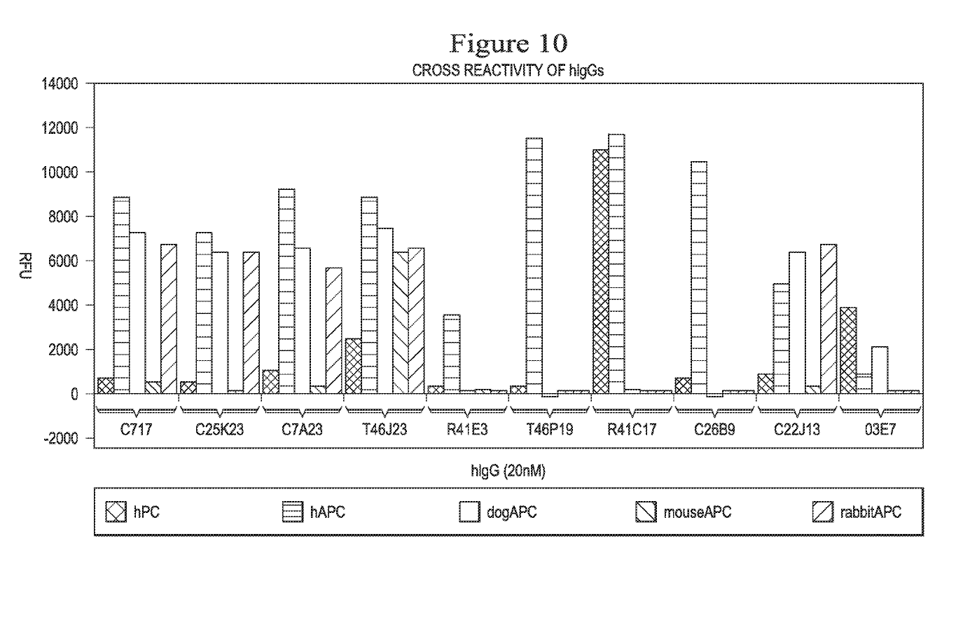

The FVa inactivation activity of human aPC can be measured by incubating human aPC (180 pM) with its physiological protein substrate FVa (1.25 nM), then adding FXa and prothrombin to the reaction mixture to form prothrombinase complex. Chromogenic peptide substrate of thrombin was added to detect the production of thrombin (FIG. 9). The readout is thrombin production (FIIa/sec). Purified factors Va (1.25 nM) were incubated with aPC (180 pM) in the presence of range of concentrations of the Fabs (1-500 nM), and the FVa activities were evaluated in the prothrombinase/tenase assay.

The influence of the Fabs on the aPC activity toward the biological substrate FVa was measured by an FXa- and a thrombin-generation assay utilizing purified FVa. In this assay, FVa at 0.16 U/ml (1.25 nM) was incubated with aPC 180 pM in assay buffer (20 mM TrisHCl, 137 nM NaCl, 10 ug/ml phospholipids, 5 mM CaCl2, 1 mg/ml BSA) in the presence or absence of antibodies. After incubation for 30 min, 25 ul mixture was transferred to wells. Subsequently, 50 ul human FXa and prothrombin in assay buffer was added to the wells and the kinetics of thrombin-mediated substrate hydrolysis monitored at 30° C. by using plate reader. As the baseline of aPC activity, in the absence of added Fab, incubation of aPC changed the readout from 0.0022 nM FIIa/sec to 0.0015 nM FIIa/sec.

Addition of the Fabs to the reaction mixtures resulted in a nearly complete inhibition of aPC-mediated proteolysis of FVa and a rapid increase in thrombin generation in a dose-dependent manner. As shown in FIG. 9, IC50 values for the inhibition of proteolysis of FVa by aPC were in the nanomolar range and were comparable for all Fabs tested. Most Fabs were more potent than the positive control Fab. R41E3 had a slower increase due to its weaker binding to human aPC. R41C17 surprisingly showed some activity in this assay. This Fab had no effect on aPC's anti-coagulant activity by aPTT or on aPC's amidolytic activity when small peptide substrate was used. These data indicate that R4117 binding epitope differs significantly from those of other Fabs.

### Example 7

### Expression and Purification of Anti-aPC IgGs

All 10 anti-aPC Fabs were converted to human IgG1 by cloning Fv sequences into human IgG1 expression vectors. Plasmids were transfected into HEK293 cells for transient expression. Antibodies were secreted into the culture medium and purified by protein A column. One high-yield antibody T46J23-hIgG1 produced 10.3 mg per 200 ml culture. Some antibodies only produced 1 mg per 200 ml. Endotoxin levels were also monitored (less than 0.01 EU/mg).

Similar to purified Fabs, all purified IgGs were characterized by a panel of functional assays to assess a) their binding specificity and binding affinity; b) their species cross-reactivity (binding to aPCs of different species origins including rabbit aPC); c) their effects on the enzymatic activity of species aPC's using amidolytic activity assay; and d) their potency of inhibiting aPC's anti-coagulant activity in the plasma clotting assay aPTT using human plasma and mouse plasma.

### Example 8

### Binding Specificity and Binding Affinity of Anti-aPC IgGs

As shown in FIG. 10, ELISA revealed that most IgG antibodies retain their binding specificity like Fabs as they preferentially bind to human aPC over human PC. On the other hand, R41C17 and 03E7 bind both human aPC and human PC. Surprisingly, T46J23 gained human PC binding after its conversion of Fab to IgG. Titration experiment by ELISA also revealed that, in general, the binding affinity of these bivalent IgG1 was increased 2-50-fold as compared to the corresponding monovalent Fabs as shown in Table 5. In particular, the low-affinity Fab R41E3 increased binding affinity almost 50-fold after Fab-IgG conversion with EC50 value of 104 nM for Fab vs. 1.76 nM for IgG. All IgGs showed high-affinity binding to human APC with EC50 values of subnanomolar and low nanomolar range. O3E7-IgG is the weakest IgG with EC50 of 16.9 nM.

TABLE 5
ELISA analysis of anti-aPC IgGs

| EC₅₀ (nM) | Direct ELISA (aPC coating) | | | |
|---|---|---|---|---|
| hIgG1 | Human aPC | Rabbit aPC | Dog aPC | Mouse aPC |
| C7A23 | 3.29 | 7 | 10.9 | |
| C7I7 | 2.66 | 2 | 2.3 | |
| C22J13 | 0.49 | 0.6 | 2.0 | |
| C25K23 | 1.25 | 2 | 3.4 | |
| C26B9 | 0.65 | | | |
| R41C17 | 0.55 | | | |
| R41E3 | 1.76 | | | |
| T46J23 | 2.18 | 7 | 1.68 | 4.6 |
| T46P19 | 0.37 | | | |
| O3E7 | 16.9 | | | |

Also shown in FIG. 10, species cross-reactivity of these IgG was investigated using (a) human, (b) rabbit, (c) dog, (d) mouse aPCs and PCs. Among 10 anti-human aPC IgGs, 5 IgGs bind to rabbit aPC with high affinity (EC₅₀=0.6-7 nM) without detectable binding to rabbit PC. These 5 IgGs also bind to dog APC with high affinity (EC₅₀=1.7-10 nM) and they did not bind to dog PC. One antibody among the 5 IgGs, T46J23, also binds to mouse aPC with EC₅₀ value of 6 nM. T46J23 did not bind to mouse PC.

### Example 9

### Effect of Anti-APC IgGs on the Enzymatic Activity of Species APC's in Buffer Using Amidolytic Activity Assay

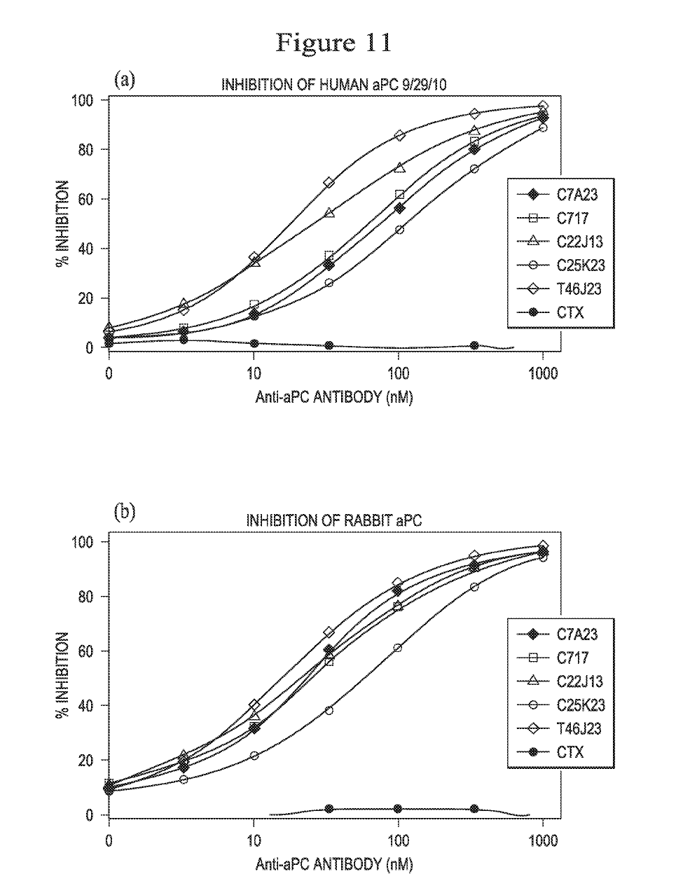

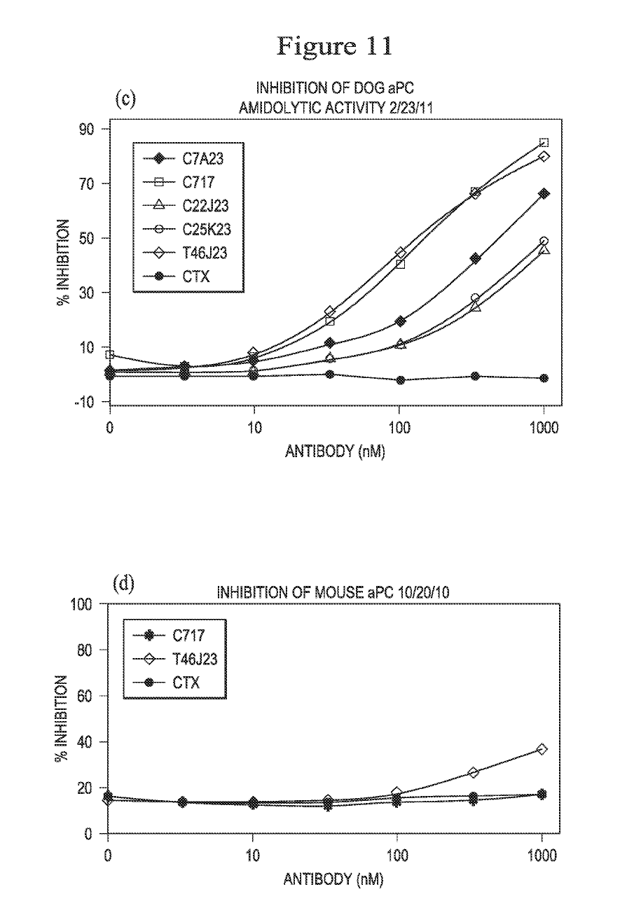

The 5 species cross-reactive IgGs were then evaluated for their effect on the amidolytic activity of species APCs (FIG. 11). In human aPC amidolytic activity assays, the negative control IgG (anti-CTX antibody) had no inhibitory effect. The 5 IgGs all inhibited human aPC in a dose-dependent manner. Their IC50 values are 18 nM for T46J23-IgG; 27 nM for C22J13; 64 nM for C7I7; 78 nM for C7A23, and 131 nM for C25K23.

In rabbit aPC amidolytic activity assays, the negative control IgG (anti-CTX antibody) had no inhibitory effect. The 5 IgGs all inhibited rabbit aPC in a dose-dependent manner. Their IC50 values are 17 nM for T46J23-IgG; 24 nM for C22J13; 29 nM for C7I7; 25 nM for C7A23, and 74 nM for C25K23.

In dog aPC amidolytic activity assays, the negative control IgG (anti-CTX antibody) had no inhibitory effect. The 5 IgGs weakly inhibited dog aPC in a dose-dependent manner. Their IC50 values are 625 nM for T46J23-IgG; 1300 nM for C22J13; 147 nM for C7I7; 49 nM for C7A23, and 692 nM for C25K23.

In mouse aPC amidolytic activity assays, only T46J23 could inhibit mouse aPC although it needs high dose (1000 nM). C7I7 and other IgGs had no effect on mouse aPC. The inhibitory effects of these antibodies on species APC activity are summarized in Table 6.

TABLE 6
ELISA and Amidolytic activity

| | Anti-aPC EC₅₀ (nM) | | | Anti-aPC IC₅₀ (nM) | | |
|---|---|---|---|---|---|---|
| | ELISA | | | Amidolytic activity | | |
| | Human | Rabbit | | Human | Rabbit | |
| hIgG1 | aPC | aPC | Dog aPC | aPC | aPC | Dog aPC |
| C7A23 | 3.3 | 7 | 10.9 | 78 | 25 | 49 |
| C7I7 | 2.7 | 2 | 2.3 | 64 | 29 | 147 |
| C22J13 | 0.5 | 0.6 | — | 27 | 24 | 1300 |
| C25K23 | 1.2 | 2 | 3.4 | 131 | 74 | 692 |
| T46J23 | 2.2 | 1.7 | 1.7 | 18 | 17 | 625 |

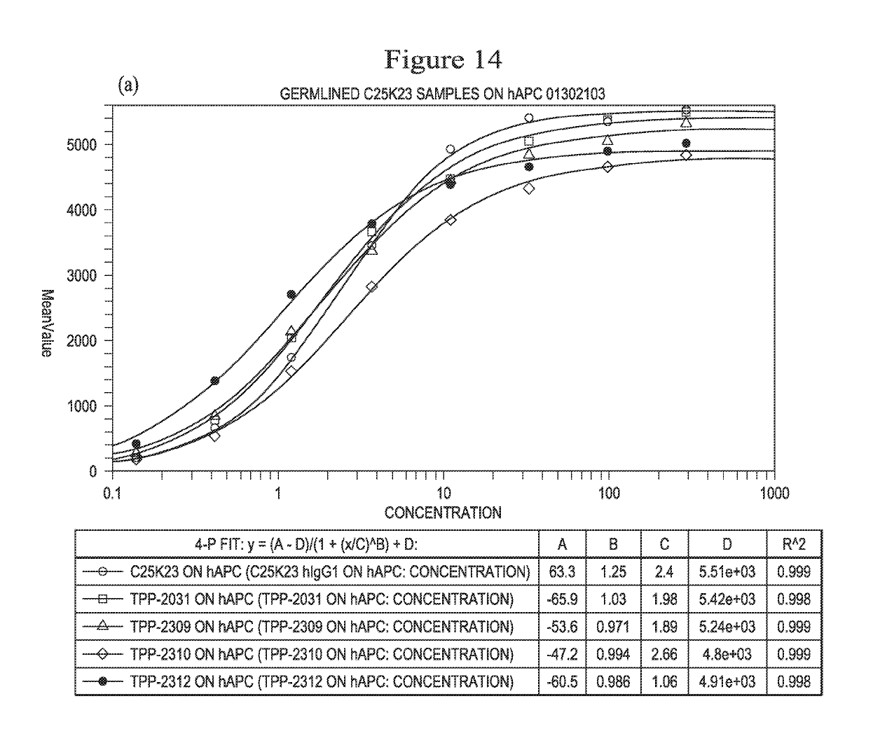

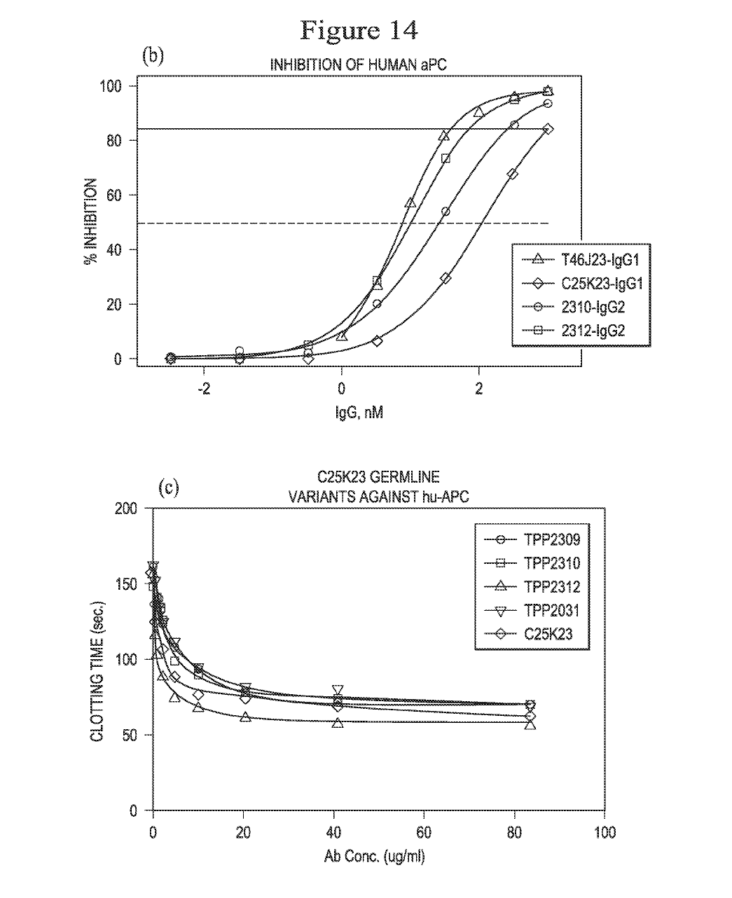

Shown in FIG. 14(b), in human aPC amidolytic activity assays, two variants of C25K23 IgG1 referred to as 2310-IgG2 and 2312-IgG2 show potent inhibition of aPC in a purified system. C25K23 IgG1 has a light chain as shown in SEQ ID NO:108 and heavy chain as shown in SEQ ID NO:109. TPP-2031 is a modified C25K23 IgG with a heavy chain comprising the modification N54G. Variant 2310 is a modified C25K23 IgG with a light chain comprising the modifications A10V, T13A, S78T, R81Q and S82A as shown in SEQ ID NO: 112 and heavy chain comprising the modification N54Q as shown in SEQ ID NO:113. Variant 2312 is a modified C25K23 IgG with a light chain comprising the modifications A10V, T13A, S78T, R81Q and S82A as shown in SEQ ID NO: 116 and heavy chain comprising the modification S56A as shown in SEQ ID NO:117. Such variants also display a high affinity to aPC as shown in FIG. 14(a). TPP-2309 is a modified C25K23 IgG1 with a light chain comprising the modifications A10V, T13A, S78T, R81Q and S82A as shown in SEQ ID NO: 110 and heavy chain comprising the modification N54G as shown in SEQ ID NO:111.

### Example 10

### Anti-aPC IgGs Inhibit aPC and Induce Clot Formation in Normal Human Plasma

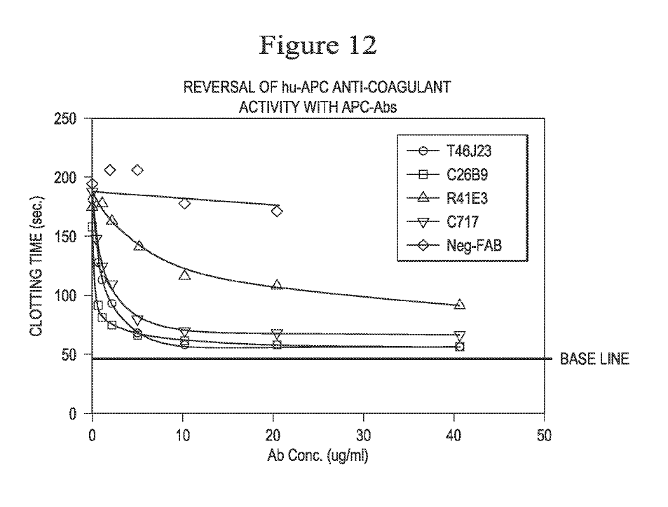

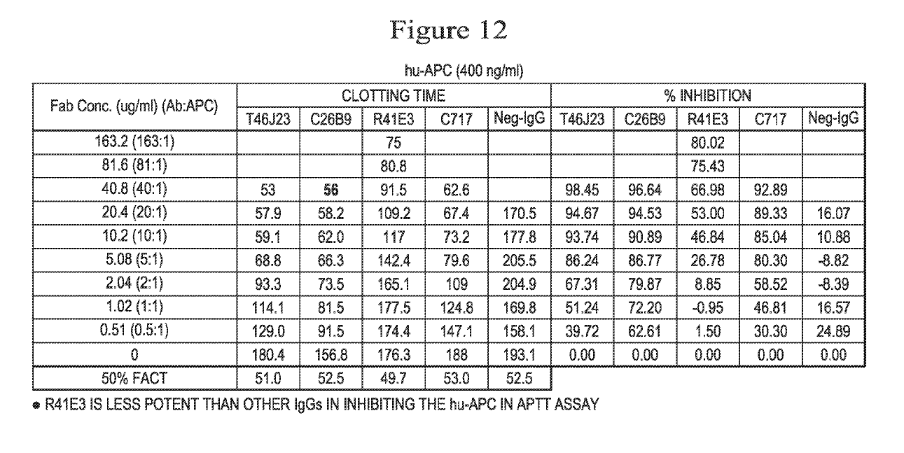

The effect of anti-aPC IgGs on aPC's anti-coagulant activity was first investigated in human plasma clotting assays (aPTT) and is shown in FIG. 12. Fifty percent (50%) human plasma had a baseline clotting time of 50-52 sec in the absence of aPC. Addition of human aPC to plasma increased clotting time to 190 sec as expected, since aPC is a well-known anti-coagulant. Pre-incubation of aPC with the negative control IgG1 (anti-CTX antibody) did not change clotting time. In contrast, pre-incubation of aPC with anti-aPC specific IgG significantly shortened the clotting time in a dose-dependent manner. At 1:1 molar ratio, both T46J23-IgG and C717-IgG at 1 ug/ml inhibited ˜50% activity of aPC (at 400 ng/ml) and shortened the clotting time from 190 to 114 sec. At 20 ug/ml, all three antibodies (T46J23, C7I7, C26B9) completely reverse the anti-coagulant activity of aPC and restored the clotting to normal. R41E3-IgG was less potent than these 3 IgGs in inhibiting aPC. R41E3 partially restored the clotting time to 75 sec and inhibited ˜80% activity of aPC at 163-fold molar excess.

The effect of modified variants of anti-aPC IgGs was also investigated in an aPTT assay as shown in FIG. 14(c). Again similar to the results in FIG. 12, pre-incubation of aPC with the modified anti-aPC specific IgG significantly shortened the clotting time in a dose-dependent manner.

### Example 11

### Anti-aPC IgGs Inhibit aPC and Induce Clot Formation in Severe Hemophilic Patient Plasma

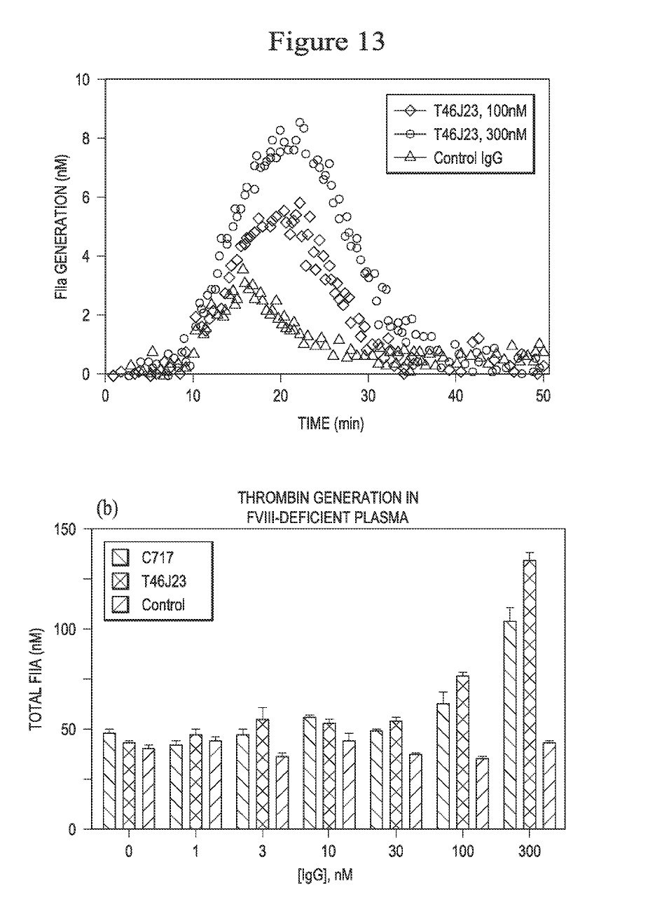

The effect of anti-APC IgGs on aPC's anti-coagulant activity was further investigated using Hemophilic patient plasma in thrombin generation assay (TGA) as shown in FIG. 13. Damages on the cells lining blood vessel (endothelial cells) results in exposure of tissue factor leading to limited amount of thrombin generation, known as extrinsic coagulation pathway. Thrombomodulin on the endothelial cells contribute to generation of aPC and its anti-coagulant activity. Severe hemophilic plasma generated only ˜50 nM total thrombin. Adding anti-aPC-antibody to the hemophilic plasma increased thrombin generation in dose dependent manner.

### Example 12

### Co-Crystal Studies

### Antibody Preparation and QC

Recombinant anti-aPC human Fabs (C25K23 and T46J23) were expressed in E. coli and purified to homogeneity by Protein A chromatography. Purified Fabs were showed to have a >90% purity and are lack of aggregation by SDS-PAGE and by analytical size exclusion chromatography. Their functions were characterized by aPC-binding assay (ELISA). Both C25K23Fab and T46J23Fab bind human aPC full-length and the Gla-domainless aPC at comparable EC₅₀ values of 2-4 nM as measured by ELISA. Ten milligrams of these Fabs were produced.

### Antigen Preparation and QC

Plasma-derived human aPC-Gla-domain-less (aPC-GD) was purchased from Enzyme Research Lab and characterized by ELISA to confirm that it can be recognized by both C25K23Fab and T46J23Fab.

### Complex Formation

For complex formation, 0.9 mg aPC-GD was mixed with 1.05 mg C25K23Fab and the reaction mixture was incubated at 4° C. for 5 hours. The mixture was loaded onto a gel filtration column to separate free Fab or free aPC-GD from the aPC-GD-Fab complex. Each fraction was collected and analyzed by SDS-PAGE under a non-reducing condition. This process was repeated three times, and the fractions containing the aPC-GD-Fab complex were pooled and concentrated to 10 mg/ml.

Crystallization of aPC-Fab complexes under different crystal growth conditions were performed to produce crystals suitable for structure determination (max. resolution <3 Å). High throughput crystallization screening kits were utilized and 2 hits were identified:
a) 0.1% n-Octyl-β-D-glucoside, 0.1M sodium citrate tribasic dihydrate PH5.5, 22% PEG 3350
b) 18% 2-propanol, 0.1M sodium citrate tribasic dihydrate PH5.5, 20% PEG 4000

### Data Collection

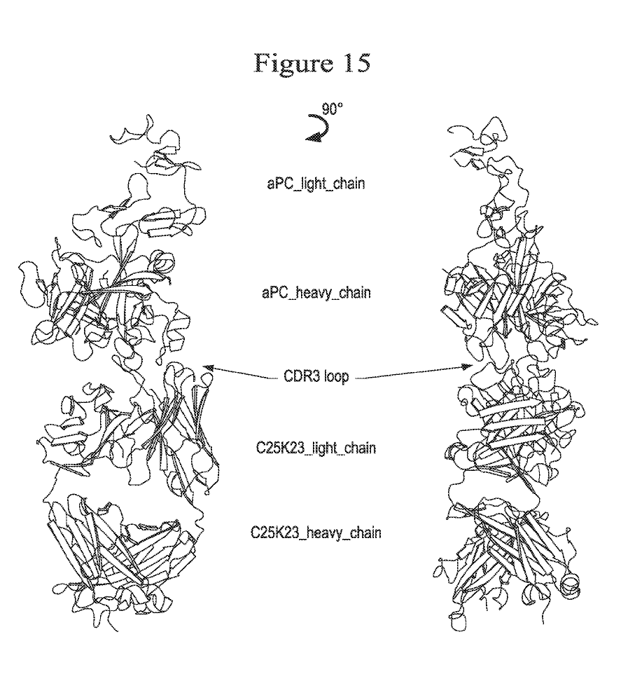

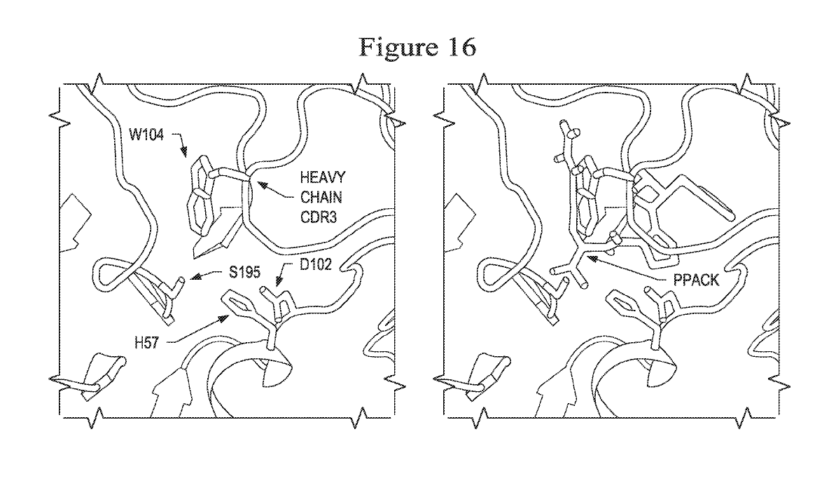

Structure determination at 2.2 angstrom resolution was successful from aPC-GD-C25K23Fab crystal diffraction image by Molecular Replacement with reported aPC and Fab X-ray structures as models (e.g. pdb code laut by Mather et al., 1996), followed by model building and refinement. Shown in FIG. 15 is a cartoon representation of the aPC and C25K23 Fab structure. As shown in FIG. 15, the C25K23 utilizes the CDR3 loop of its heavy chain to contact the aPC catalytic domain. Very significantly, as shown in FIG. 16, the side chain of W104 from the C25K23 inserts into the catalytic pocket of aPC, having steric overlap with a previously reported aPC inhibitor (tri-peptide inhibitor PPACK).

From this structure, it was determined that the epitope of aPC bound by the antibody is in the heavy chain of aPC. Contacting residues between the aPC heavy chain and Fab include aPC residues D60, K96, S97, T98, T99, E170, V171, M172, 5173, M175, A190, S195, W215, G216, E217, G218, and G218.

Specifically for Fab C25K23, it was determined that the paratope comprises residues S31, Y32, W53, R57, R101, W104, R106, F107, W110 of the heavy chain shown in SEQ ID NO:18 and K55 of the light chain shown in SEQ ID NO:8.

### Example 13

### Active-Site Binding

An irreversible active-site inhibitor, biotin-PPACK, was used to occupy the active site of human aPC, see FIG. 16. Biotin-PPACK-hAPC or human aPC was coated onto a maxisorp 96-well plate. Anti-aPC antibodies (Fab and IgG) were serial-diluted at 1:3 from 20 nM to 0.007 nM and added to coated wells and incubated for 1 h at room temperature. The bound anti-aPC-Fab or anti-aPC IgG was detected by HRP-conjugated anti-human or anti-mouse Fab antibody followed by incubation with fluorogenic substrates (amplex red and H2O2) to produce fluorescent signals (RFU). The plate was read by Gemini EM fluorescence microplate reader (Molecular Devices, Sunnyvale, Calif.). The RFUs at 20 nM antibody concentration were presented as mean of triplicate wells (+/−SD) in the bar graph.

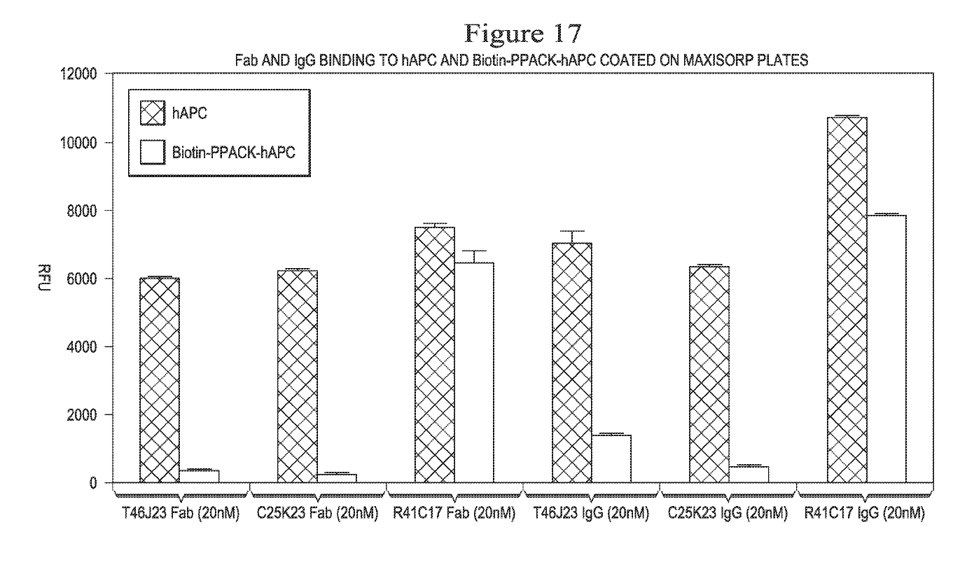

As shown in FIG. 17, at least two types of antibodies were identified from the library. First those that are active-site directed including T46J23 (Fab and hIgG) and C25K23 (Fab and hIgG) which no longer bound to biotin-PPACK-hAPC (the active site-blocked hAPC). Second, those that are non-active-site directed including R41C17 which is believed to be an anti-Gla-domain antibody. These data provide solid evidence for active-site binding of T46J23 and C25K23 on human aPC and explain the functional characteristics of these antibodies, i.e. complete blockade of hAPC activities.

While the present embodiments have been described with reference to the specific embodiments and examples, it should be understood that various modifications and changes can be made and equivalents can be substituted without departing from the true spirit and scope of the claims appended hereto. The specification and examples are, accordingly, to be regarded in an illustrative rather than in a restrictive sense. Furthermore, the disclosure of all articles, books, patent applications and patents referred to herein are incorporated herein by reference in their entireties.

1. An isolated monoclonal antibody, wherein said antibody binds to activated Protein C and inhibits anticoagulant activity but has minimal binding to unactivated Protein C, wherein said antibody comprises a heavy chain variable region comprising an amino acid sequence selected from the group consisting of SEQ ID Nos: 14, 15, 17, 18, 19, 21, 22, 23, 109, 111, 113, 115, 117 and 119.

2. An isolated monoclonal antibody, wherein said antibody binds to activated Protein C and inhibits anticoagulant activity but has minimal binding to unactivated Protein C, wherein said antibody comprises a light chain variable region comprising an amino acid sequence selected from the group consisting of SEQ ID Nos: 4, 5, 7, 8, 9, 11, 12, 13, 108, 110, 112, 114, 116 and 118.

3. The isolated monoclonal antibody of claim 1 further comprising a light chain variable region comprising an amino acid sequence selected from the group consisting of SEQ ID Nos: 4, 5, 7, 8, 9, 11, 12, 13, 108, 110, 112, 114, 116 and 118.

4. The isolated monoclonal antibody of claim 3, wherein the antibody comprises heavy and light chain variable regions comprising:
a) a heavy chain variable region having an amino acid sequence of SEQ ID NO: 14 and a light chain variable region having an amino acid sequence of SEQ ID NO: 4;
b) a heavy chain variable region having an amino acid sequence of SEQ ID NO: 15 and a light chain variable region having an amino acid sequence of SEQ ID NO: 5;
c) a heavy chain variable region having an amino acid sequence of SEQ ID NO: 17 and a light chain variable region having an amino acid sequence of SEQ ID NO: 7;
d) a heavy chain variable region having an amino acid sequence of SEQ ID NO: 18 and a light chain variable region having an amino acid sequence of SEQ ID NO: 8;
e) a heavy chain variable region having an amino acid sequence of SEQ ID NO: 19 and a light chain variable region having an amino acid sequence of SEQ ID NO: 9;
f) a heavy chain variable region having an amino acid sequence of SEQ ID NO: 21 and a light chain variable region having an amino acid sequence of SEQ ID NO: 11;
g) a heavy chain variable region having an amino acid sequence of SEQ ID NO: 22 and a light chain variable region having an amino acid sequence of SEQ ID NO: 12;
h) a heavy chain variable region having an amino acid sequence of SEQ ID NO: 23 and a light chain variable region having an amino acid sequence of SEQ ID NO: 13;
i) a heavy chain variable region having an amino acid sequence of SEQ ID NO: 109 and a light chain variable region having an amino acid sequence of SEQ ID NO: 108;
j) a heavy chain variable region having an amino acid sequence of SEQ ID NO: 111 and a light chain variable region having an amino acid sequence of SEQ ID NO: 110;
k) a heavy chain variable region having an amino acid sequence of SEQ ID NO: 113 and a light chain variable region having an amino acid sequence of SEQ ID NO: 112;
l) a heavy chain variable region having an amino acid sequence of SEQ ID NO: 115 and a light chain variable region having an amino acid sequence of SEQ ID NO: 114;
m) a heavy chain variable region having an amino acid sequence of SEQ ID NO: 117 and a light chain variable region having an amino acid sequence of SEQ ID NO: 116; and
n) a heavy chain variable region having an amino acid sequence of SEQ ID NO: 119 and a light chain variable region having an amino acid sequence of SEQ ID NO: 118.

5. An isolated monoclonal antibody, wherein said antibody binds to activated Protein C and inhibits anticoagulant activity but has minimal binding to unactivated Protein C, wherein said antibody comprises a CDR3 comprising an amino acid sequence selected from the group consisting of SEQ ID NOs: 94, 95, 97, 98, 99, 101, 102 and 103.

6. The isolated monoclonal antibody of claim 5, wherein the antibody further comprises (a) a CDR1 comprising an amino acid sequence selected from the group consisting of SEQ ID NOs: 74, 75, 77, 78, 79, 81, 82 and 83, (b) a CDR2 comprising an amino acid sequence selected from the group consisting of SEQ ID NOs: 84, 85, 87, 88, 89, 91, 92 and 93, or (c) both a CDR1 comprising an amino acid sequence selected from the group consisting of SEQ ID NOs: 74, 75, 77, 78, 79, 81, 82 and 83 and a CDR2 comprising an amino acid sequence selected from the group consisting of SEQ ID NOs: 84, 85, 87, 88, 89, 91, 92 and 93.

7. An isolated monoclonal antibody, wherein said antibody binds to activated Protein C and inhibits anticoagulant activity but has minimal binding to unactivated Protein C, wherein said antibody comprises a CDR3 comprising an amino acid sequence selected from the group consisting of SEQ ID NOs: 64, 65, 67, 68, 69, 71, 72 and 73.

8. The isolated monoclonal antibody of claim 7, wherein the antibody further comprises (a) a CDR1 comprising an amino acid sequence selected from the group consisting of SEQ ID NOs: 44, 45, 47, 48, 49, 51, 52 and 53, (b) a CDR2 comprising an amino acid sequence selected from the group consisting of SEQ ID NOs: 54, 55, 57, 58, 59, 61, 62 and 63, or (c) both a CDR1 comprising an amino acid sequence selected from the group consisting of SEQ ID NOs: 44, 45, 47, 48, 49, 51, 52 and 53 and a CDR2 comprising an amino acid sequence selected from the group consisting of SEQ ID NOs: 54, 55, 57, 58, 59, 61, 62 and 63.

9. The isolated monoclonal antibody of claim 5, wherein the antibody further comprises a CDR3 comprising an amino acid sequence selected from the group consisting of SEQ ID NOs: 64, 65, 67, 68, 69, 71, 72 and 73.

10. The isolated monoclonal antibody of claim 9, wherein the antibody further comprises (a) a CDR1 comprising an amino acid sequence selected from the group consisting of SEQ ID NOs: 74, 75, 77, 78, 79, 81, 82 and 83, (b) a CDR2 comprising an amino acid sequence selected from the group consisting of SEQ ID NOs: 84, 85, 87, 88, 89, 91, 92 and 93, (c) a CDR1 comprising an amino acid sequence selected from the group consisting of SEQ ID NOs: 44, 45, 47, 48, 49, 51, 52 and 53, and (d) a CDR2 comprising an amino acid sequence selected from the group consisting of SEQ ID NOs: 54, 55, 57, 58, 59, 61, 62 and 63.

11. The antibody of claim 4, wherein the antibody comprises heavy and light chain variable regions comprising:
a) a light chain variable region comprising an amino acid sequence comprising SEQ ID NOs: 44, 54, and 64 and a heavy chain variable region comprising an amino acid sequence comprising SEQ ID NOs: 74, 84, and 94;
b) a light chain variable region comprising an amino acid sequence comprising SEQ ID NOs: 45, 55, and 65 and a heavy chain variable region comprising an amino acid sequence comprising SEQ ID NOs: 75, 85, and 95;
c) a light chain variable region comprising an amino acid sequence comprising SEQ ID NOs: 47, 57, and 67 and a heavy chain variable region comprising an amino acid sequence comprising SEQ ID NOs: 77, 87, and 97;
d) a light chain variable region comprising an amino acid sequence comprising SEQ ID NOs: 48, 58, and 68 and a heavy chain variable region comprising an amino acid sequence comprising SEQ ID NOs: 78, 88, and 98;
e) a light chain variable region comprising an amino acid sequence comprising SEQ ID NOs: 49, 59, and 69 and a heavy chain variable region comprising an amino acid sequence comprising SEQ ID NOs: 79, 89, and 99;
f) a light chain variable region comprising an amino acid sequence comprising SEQ ID NOs: 51, 61, and 71 and a heavy chain variable region comprising an amino acid sequence comprising SEQ ID NOs: 81, 91, and 101;
g) a light chain variable region comprising an amino acid sequence comprising SEQ ID NOs: 52, 62, and 72 and a heavy chain variable region comprising an amino acid sequence comprising SEQ ID NOs: 82, 92, and 102; and
h) a light chain variable region comprising an amino acid sequence comprising SEQ ID NOs: 53, 63, and 73 and a heavy chain variable region comprising an amino acid sequence comprising SEQ ID NOs: 83, 93, and 103.

12. The isolated monoclonal antibody of claim 4 further comprising one or more amino acid modifications.

13. The isolated monoclonal antibody of claim 11 further comprising one or more amino acid modifications.

14. An isolated monoclonal antibody, wherein said antibody binds to activated Protein C and inhibits anticoagulant activity but has minimal binding to unactivated Protein C, wherein said antibody comprises a light chain variable region comprising an amino acid sequence of SEQ ID NO:8, wherein said amino acid sequence comprises one or more amino acid modifications.

15. The isolated monoclonal antibody of claim 13, wherein the modification is a substitution.

16. The isolated monoclonal antibody of claim 14, wherein the substitution is a position selected from the group consisting of A10, T13, G52, N53, N54, R56, P57, S58, S78, R81, S82, Q91, Y93, S95, S96, L97, S98, G99, S100 and V101.

17. The isolated monoclonal antibody of claim 15, wherein the substitution is selected from the group consisting of A10V, T13A, G52S, G52Y, G52H, G52F, N53G, N54K, N54R, R56K, P57G, P57W, P57N, S58V, S58F, S58R, S78T, R81Q, S82A, Q91R, Q91G, Y93W, S95F, S95Y, S95G, S95W, S95E, S96G, S96A, S96Y, S96W, S96R, L97M, L97G, L97R, L97V, S98L, S98W, S98V, S98R, G99A, G99E, S100A, S100V, V101Y, V101L and V101E.

18. An isolated monoclonal antibody, wherein said antibody binds to activated Protein C and inhibits anticoagulant activity but has minimal binding to unactivated Protein C, wherein said antibody comprises a heavy chain variable region comprising an amino acid sequence of SEQ ID NO:18, wherein said amino acid sequence comprises one or more amino acid modifications.

19. The isolated monoclonal antibody of claim 18, wherein the modification is a substitution.

20. The isolated monoclonal antibody of claim 19, wherein the substitution is a position selected from the group consisting of N54 and S56.

21. The isolated monoclonal antibody of claim 20, wherein the substitution is selected from the group consisting of N54G, N54Q, N54A, S56A and S56G.

22. An isolated monoclonal antibody, wherein said antibody binds to activated Protein C and inhibits anticoagulant activity but has minimal binding to unactivated Protein C, wherein said antibody comprises a light chain variable region comprising an amino acid sequence of SEQ ID NO:12, wherein said amino acid sequence comprises one or more amino acid modifications.

23. The isolated monoclonal antibody of claim 22, wherein the modification is a substitution.

24. The isolated monoclonal antibody of claim 23, wherein the substitution is a position selected from the group consisting of T25, D52, N53, N54, N55, D95, N98 and G99.

25. The isolated monoclonal antibody of claim 24, wherein the substitution is selected from the group consisting of T25S, D52Y, D52F, D52L, D52G, N53C, N53K, N53G, N54S, N55K, D95G, N98S, G99H, G99L and G99F.

26. An isolated monoclonal antibody that binds to an epitope of human activated Protein C (human aPC, SEQ ID NO:3), wherein said epitope comprises residues from a heavy chain of human aPC.

27. An isolated monoclonal antibody that binds to an epitope of human activated Protein C (human aPC, SEQ ID NO:3), wherein said epitope comprises 5195 of SEQ ID NO:3.

28. An isolated monoclonal antibody that binds to an epitope of human activated Protein C, wherein said epitope comprises one or more residues selected from the group consisting of D60, K96, S97, T98, T99, E170, V171, M172, 5173, M175, A190, 5195, W215, G216, E217, G218, and G218 of SEQ ID NO:3.

29. An isolated monoclonal antibody that binds to the active site of activated Protein C.

30. An isolated monoclonal antibody, wherein said antibody binds to activated Protein C and inhibits anticoagulant activity but has minimal binding to unactivated Protein C, wherein said antibody is a fully human antibody.

31. The isolated monoclonal antibody of claims 1-30, wherein the antibody is selected from the group consisting of an IgG1, an IgG2, an IgG3, an IgG4, an IgM, an IgA1, an IgA2, a secretory IgA, an IgD, an IgE antibody, and antibody fragment.

32. The isolated monoclonal antibody of claims 1-30, wherein the antibody binds to human activated Protein C.

33. The isolated monoclonal antibody of claim 32, wherein the antibody further binds to a non-human species of activated Protein C.

34. The antibody of claims 1-30, wherein blood clotting time in the presence of the antibody is shortened.

35. An antibody which would compete with the antibody of claim 1-30.

36. A pharmaceutical composition comprising a therapeutically effective amount of the monoclonal antibody of any of claims 1-30 and a pharmaceutically acceptable carrier.

37. A method for treating genetic or acquired deficiencies or defects in coagulation comprising administering a therapeutically effective amount of the pharmaceutical composition of claim 36 to a patient.

38. A method for treating coagulopathy comprising administering a therapeutically effective amount of the pharmaceutical composition of claim 36 to a patient.

39. The method of claim 38, wherein the coagulopathy is hemophilia A, B or C.

40. The method of claim 38, wherein the coagulopathy is selected from the group consisting of trauma-induced coagulopathy or severe bleeding patients.

41. The method of claim 38, further comprising administering a clotting factor.

42. The method of claim 41, wherein the clotting factor is selected from the group consisting of Factor VIIa, Factor VIII or Factor IX.

43. A method for shortening bleeding time comprising administering a therapeutically effective amount of the pharmaceutical composition of claim 36 to a patient.

44. An isolated nucleic acid molecule encoding an antibody that binds to activated Protein C and inhibits anticoagulant activity but has minimal binding to unactivated Protein C, wherein the antibody comprises a heavy chain variable region comprising an amino acid sequence selected from the group consisting of SEQ ID Nos: 14, 15, 17, 18, 19, 21, 22 and 23.

45. An isolated nucleic acid molecule encoding an antibody that binds to activated Protein C and inhibits anticoagulant activity but has minimal binding to unactivated Protein C, wherein the antibody comprises a light chain variable region comprising an amino acid sequence selected from the group consisting of SEQ ID Nos: 4, 5, 7, 8, 9, 11, 12 and 13.
